(19) United States
(12) Patent Application Publication
(10) Pub. No.: US 2010/0152435 A1
(43) Pub. Date: Jun. 17, 2010

(54) COMPOSITIONS AND PROCESSES FOR THE SELECTIVE CATALYTIC OXIDATION OF ALCOHOLS

(76) Inventors: Jonathan A. Stapley, Bellevue, WA (US); Setrak K. Tanielyan, Maplewood, NJ (US); Robert L. Augustine, Livingston, NJ (US); Norman M. Marin Astorga, Newark, NJ (US); Gabriela D. Alvez Manoli, Newark, NJ (US)

Correspondence Address: INDIANAPOLIS OFFICE 27879;BRINKS HOFER GILSON & LIONE, CAPITAL CENTER, SUITE 1100, 201 NORTH ILLINOIS STREET, INDIANAPOLIS, IN, 46204-4220, US

(21) Appl. No.: 12/530,927

(22) PCT Filed: Mar. 14, 2008
(86) PCT No.: PCT/US08/57102

§ 371 Date: Feb. 10, 2010

Related U.S. Application Data
(60) Provisional application No. 60/906,923, filed on Mar. 14, 2007.
(60) Provisional application No. 60/906,921, filed on Mar. 14, 2007.

(51) Int. Cl. C07H 1/00; C08B 37/00; C07C 61/08

(52) U.S. Cl. 536/123.12; 536/124; 562/400

(57) ABSTRACT

Processes for oxidation of primary alcohols or aldehydes into the corresponding carboxylic acids are provided herein, including processes for the aerobic catalytic oxidation of a hydroxyl moiety pendant to a cyclic carbohydrate to a carboxylic acid in a manner that preserves the cyclic carbohydrate structure. The oxidation processes may be performed in the absence of a transition metal catalyst, halogenated solvent and a hypochlorite reagent. The processes and compositions with and without a bromide source are provided. A liquid reaction media comprising a carboxylic acid and a catalyst composition may be combined with the reactant alcohol substrate to form a reaction media which can be pressurized at constant volume with an oxygen-containing gas under conditions of temperature and constant pressure within a reaction vessel to selectively oxidize the reactant substrate to form a carboxylic acid product. Preferably, the reactant alcohol substrate is a cyclic carbohydrate having a pendant primary or secondary alcohol that is converted to a carboxylic acid by the selective oxidation process, such as the conversion of an alkyl-glucopyranoside to a corresponding alkyl-glucuronic acid.

### RELATED APPLICATIONS

This application claims the benefit of U.S. provisional patent application 60/906,921 (filed Mar. 14, 2007 and entitled “CATALYTIC OXIDATION OF ALCOHOLS”) and U.S. provisional patent application Ser. No. 60/906,923 (filed Mar. 14, 2007 and entitled “CATALYTIC OXIDATION OF CARBOHYDRATES”), both of which are incorporated herein by reference in their entirety.

### TECHNICAL FIELD

The present invention relates to processes and catalysts for the selective oxidation of alcohols, including selective oxidation of primary alcohol groups of carbohydrates.

### BACKGROUND

Processes for the oxidation of alcohols are advantageous for many applications. For instance, selective oxidation of carbohydrate molecules, such as alcohols pendant to sugar alcohols, may be useful for the production of carbohydrate derivatives useful as metal chelating agents, viscosifiers, carrier materials, stabilizers, and components in superabsorbent polymers. Processes for the selective oxidation of primary alcohols in carbohydrates that preserve a cyclic structure, such as a pyranose or furanose ring structure, during the oxidation of a primary alcohol pendant to the ring structure are particularly advantageous for certain applications, such as the production of carbohydrate derivative molecules useful, for example, as food additives. Carbohydrates frequently occur in nature as long chain polymers of simple sugars, and include monomeric, oligomeric, and polymeric carbohydrate compounds having a primary hydroxyl group available for reaction, including starch, cellulose, sucrose, glucosides, and fructosides. Oxidation of alcohol moieties pendant to carbohydrate molecules to carboxylic acid moieties may be useful for introducing functionalities into the molecule, such as for adjusting solubility, reactivity or for providing an anchor for coupling reactions with other molecules. Processes for the oxidation of carbohydrates at a primary hydroxyl moiety bound to a pyranose or furanose ring without altering the ring structure may be particularly desirable for certain applications.

Selective oxidation methods for oxidation of primary alcohols pendant to carbohydrate molecules may include either air or oxygen as primary oxidants in combination with catalyst systems comprising stable nitroxyl radicals and transition metal salts as co-catalysts. Commonly used co-catalysts include: (NH₄)₂Ce(NO₃)₆ (Kim, S. S.; Jung, H. C. Synthesis 2003, 14, 2135-2137), CuBr₂-2,2′-bipiridine complex (Gamez, P; Arends, I.W.C.E.; Reedijk, J.; Sheldon, R. A. Chem. Commun. 2003, 19, 2414-2415), RuCl2(PPh₃)₃ (Inokuchi, T.; Nakagawa, K.; Torii, S. Tetrahedron Letters, 36, 3223-3226 and Dijksman, A.; Marino-Gonzalez, A.; Payeras, A. M.; Arends, I.W.C.E.; Sheldon, R. A. J. Am. Chem. Soc. 2001, 123, 6826-6833), Mn(NO₃)₂—Co(NO₃)₂ and Mn(NO₃)₂—Cu(NO₃)₂ (Cecchetto, A.; Fontana, F.; Minisci, F.; Recupero, F. Tetrahedron Letters, 2001, 42, 6651-6653), CuCl in ionic liquid [bmim][PF₆] (Imtiaz, A. A; Gree, R. Organic Letters 2002, 4, 1507-1509). However, since these methods typically require the use of transition metal complexes, such oxidation processes can be expensive to run and properly dispose of the catalytic materials.

Hu et al. disclose a procedure for aerobic oxidation of primary and secondary alcohols utilizing a TEMPO based catalyst system, free of any transition metal co-catalyst (Liu, R.; Liang, X.; Dong, C.; Hu, X. J. Am. Chem. Soc. 2004, 126, 4112-4113). The procedure disclosed by Hu et al. uses a mixture of TEMPO (1 mol %), sodium nitrite (4-8 mol %) and bromine (4 mol %) as the active catalyst system. The oxidation takes place at temperatures between 80-100° C. and at an air pressure of 4 bars. However, the process is only successful with activated alcohols. With benzyl alcohol, quantitative conversion is achieved after 1-2 h of reaction time. In the case of non-activated aliphatic alcohols (such as 1-octanol) or a cyclic alcohols (cyclohexanol), the air pressure needs to be raised up to 9 bar and 4-5 h of reaction time was necessary to reach complete conversion. Disadvantageously, this oxidation procedure again depends on dichloromethane as a solvent, which is a major obstacle for an industrial application of the method and is inapplicable to most carbohydrates. Furthermore, elemental bromine as an oxidant may be difficult to handle on a large scale due to its high vapor pressure, and severe corrosion when applied in standard steel apparatus. Other disadvantages of this method are the rather low substrate concentration in the solvent used and the observed formation of bromination by-products.

Another approach to selectively oxidizing primary alcohol groups uses a nitroxyl compound as an intermediary oxidizing agent and hypochlorite as the terminal oxidant. Anelli et al., J. Org. Chem. 52, 2559 (1987), and 54, 2970 (1989), reported the oxidation of alcohols and diols with sodium hypochlorite, potassium bromide and 2,2,6,6-tetramethylpiperidinyloxy (TEMPO) or 4-methoxy-TEMPO in a two-phase solvent system (dichloromethane and water) at pH 9.5. Davis and Flitsch, Tetrahedron Lett. 34, 1181-1184 (1993), reported the oxidation of mono-saccharides wherein the non-primary hydroxyl groups are partly protected, using the same oxidation system. Advantageously, the TEMPO oxidations can also be carried out in non-toxic media, especially aqueous media. DE-4209869 discloses the oxidation of alkyl polyglucosides and other compounds having primary alcohol functions with hypochlorite and TEMPO in aqueous suspension at pH 8-9. De Nooy et al (WO 95/07303 and Recl. Tray. Chim. Pays-Bas 113 (1994) 165-166) have described the oxidation of polysaccharides using TEMPO and a hypohalite in the presence of a catalytic amount of a TEMPO or a related nitroxyl radical in an aqueous reaction medium at a pH of between 9 and 13. Similarly, DE-19746805 describes the oxidation of starch with TEMPO, hypochlorite or chlorine, and bromide at pH 7-9. WO 99/23117 and WO 99/23240 describe the oxidation of cellulose and starch, respectively, with TEMPO and an oxidative enzyme (laccase) and oxygen at pH 4-9 resulting in products containing low numbers of carbaldehyde and carboxyl groups. Further research has been reported by Isogai and Kato Cellulose 1998, 5, 153-164, and Chang and Robyt, J. Carbohydrate Chem. 15, 819-830 (1996). One disadvantage of using sodium hypochlorite or any other hypohalite as stoichiometric oxidant is that per mol of alcohol oxidized during the reaction one mole of halogenated salt is formed. Furthermore, the use of hypohalites very frequently leads to the formation of undesirable halogenated by-products thus necessitating further purification of the oxidation product. A review of methods based on the TEMPO based oxidations is found, for example, in Synthesis, 1996, 1153-1174; Topics in Catalysis 2004, 27, 49-66; Acc. Chem. Res. 2002, 35, 774-781.

Kochkar et el. (J. Catalysis 194, 343-351 (2000)) described the TEMPO-mediated oxidation of α-methyl-D-glucoside (α-MDG), 1,2-propanediol, saccharose and starch with ammonium peroxodisulfate in the presence of a supported sliver catalyst in water at pH 9.5 at 25° C. The oxidation of α-MDG and propanediol was disclosed at 78% conversion and 99% selectivity for oxidation of primary hydroxyl group for α-MDG and 90% conversion and 75% selectivity for propanediol. However, the oxidation of saccharose was mediocre (20% conversion) and oxidation of starch was unsuccessful (less than 1% conversion). In the absence of the silver catalyst, the TEMPO oxidation of α-MDG with peroxodisulfate was poor (9% conversion), while replacing peroxodisulfate by Oxone® (2 KHSO₅.KHSO₄.K₂SO₄) in the presence of silver resulted in only 6% conversion. The oxidation of benzyl alcohol and other alcohols with TEMPO and Oxone® in organic solvent to produce aldehydes end ketones was described by Bolm et al. (Org. Lett 2. 1173-1175 (2000)).

U.S. Pat. No. 6,518,419 (Van Der Lugt, et al.), filed Nov. 7, 2000, describes a process for oxidizing a primary alcohol using an oxidizing agent in the presence of a catalytic amount of a di-tertiary-alkyl nitroxyl, comprising subjecting the primary alcohol to a peracid or a precursor thereof as the oxidizing agent and to said di-tertiary nitroxyl, in the presence of 0.1-40 mol % of halide, with respect to the primary alcohol. The oxidation of carbohydrates containing primary hydroxyl groups results in the corresponding carbohydrates containing aldehydes and/or carboxylic acids with intact ring systems. Examples include α-1,4-glucan-6-aldehydes, fructan-6-aldehydes and β-2,6-fructan-1-aldehydes, with the corresponding carboxylic acids.

Recently, transition-metal free catalytic methods of selectively oxidizing a primary or secondary alcohol have been disclosed by Tanielyan et al. in U.S. Pat. No. 7,030,279, issued Apr. 18, 2006, entitled “Process for Transition Metal Free Catalytic Aerobic Oxidation of Alcohols under Mild Conditions using Free Nitroxyl Radicals,” and filed Dec. 23, 2004. Accordingly, an alcohol can be oxidized by a process in which a primary or secondary alcohol is reacted with an oxygen-containing gas in the presence of a catalyst composition containing (i) a free nitroxyl radical derivative, (ii) a nitrate source, (iii) a bromide source, and (iv) a carboxylic acid, thereby obtaining an aldehyde or a ketone.

What are needed are improved methods for the selective oxidation of primary alcohol moieties of carbohydrates to carboxylic acid moieties, including methods for the selective oxidation of primary hydroxyl moieties pendant to a cyclic carbohydrate compound without oxidizing secondary hydroxyl moieties forming the cyclic carbohydrate compound. In particular, methods for oxidation that can be performed using molecular oxygen or air as an oxidant in the absence of catalytic reagents comprising a transition metal or a hypochlorite agent would be desirable. Furthermore, catalytic compositions and methods are needed for the selective oxidation of primary or secondary alcohol moieties on carbohydrates to carboxylic acid moieties in the absence of a bromide source, including methods for the selective oxidation of primary or secondary hydroxyl moieties pendant to a cyclic carbohydrate substrate.

### SUMMARY

This disclosure provides catalytic compositions, systems and processes for the oxidation of primary alcohol moieties or aldehyde moieties to carboxylic acids. The compositions, systems and methods are generally applicable to the oxidation of a variety of reactant substrates—including alkanols, glycols and carbohydrates—but are preferably applied to the oxidation of carbohydrate substrates, such as sucrose or an alkylglucopyranoside. Preferably, the substrate is a sugar alcohol oxidized to a carboxylic acid derivative by oxidation of a primary or secondary alcohol pendant to a cyclic carbohydrate structure. Preferred embodiments provide methods and catalytic systems for the aerobic catalytic oxidation of a primary hydroxyl moiety pendant to a cyclic carbohydrate sugar alcohol to convert the hydroxyl moiety to a carboxylic acid in a manner that preserves the cyclic carbohydrate structure, without oxidizing secondary alcohol moieties in the carbohydrate ring structure. The preferred oxidation processes may be performed by contacting a reactant substrate with an oxygen source in a fluid reaction medium containing a catalyst composition under conditions of temperature and pressure effective to oxidize a primary alcohol moiety or aldehyde moiety to a carboxylic acid. Preferably, the process uses molecular oxygen or air as an oxidant and may be performed at desirably high substrate to catalyst molar ratios. Importantly, the oxidation process may be performed in the absence of a transition metal catalyst, halogenated solvent, and a hypochlorite reagent. The oxidation methods may also be performed in the absence of a bromide source, and the catalytic compositions may be formulated without including a bromide source.

In a first embodiment, catalytic compositions are provided. The catalytic compositions may be combined with a reactant substrate under suitable conditions of temperature and pressure to selectively oxidize an alcohol or an aldehyde moiety, of the reactant substrate to a carboxylic acid. Preferably, the catalytic composition comprises a nitroxyl radical, a nitrogen-containing co-catalyst and an organic acid. The nitroxyl radical is preferably a stable free nitroxyl radical or an oxoammonium derivative that is desirably stable at room temperature for a period of about one week or longer in the presence of oxygen. One particularly preferred nitroxyl radical is 4-acetamino-2,2,6,6-tetramethyl-piperidine-N-oxyl (AA-TEMPO). Preferably, the molar ratio of the nitroxyl radical to the substrate is minimized, typically between 0.001 and 10 mol %, preferably about 2 mol % or less. Particularly preferred catalytic compositions have a molar ratio of the nitroxyl radical to the nitrogen-containing co-catalyst of about 1:1. The nitrogen-containing co-catalyst preferably includes one or more compounds selected from the group consisting of: a nitrate source, nitric oxide (NO) and nitrogen dioxide (NO₂). Examples of nitrate sources include nitric acid, ammonium nitrate, alkyl ammonium nitrate and any alkali or alkaline-earth nitrate. In one aspect, the nitrogen-containing co-catalyst comprises two or more nitrogen-containing co-catalysts. For example, the catalytic composition may include both nitrogen dioxide and a nitrate source, such as nitric acid. The catalytic composition preferably includes the one or more nitrogen-containing co-catalyst in an amount of about 0.01-0.2 mol % of the nitrogen-containing co-catalyst with respect to the substrate. The catalyst composition may include the nitroxyl radical and nitric acid in a molar ratio of about 2:1. Preferably, the molar ratio of the substrate to the nitroxyl radical in the reaction media is at least about 40:1, more preferably about 100:1 and most preferably about 120:1, 150:1, 190:1, or greater.

The organic acid in the catalytic composition is preferably a carboxylic acid that is different from the carboxylic acid produced by the oxidation of the reactant substrate. The carboxylic acid in the catalytic composition is typically acetic acid, although other carboxylic acids may be used.

In one aspect of the first embodiment, the catalytic composition further includes a bromide source. The bromide source may be any suitable bromide-containing species, including N-Bromosuccinimide (NBS) or HBr. The bromide source is preferably present in trace amounts, typically about 0.01-1.00 mol % with respect to the substrate, and preferably about 0.01-0.2 mol % with respect to the substrate. Preferred catalytic compositions have molar ratios of about 1.0:0.1 between the nitroxyl radical and the bromide source and/or between the nitrogen-containing co-catalyst and the bromide source. Alternatively, in a second aspect of the first embodiment, catalytic compositions are provided that do not include a bromide source.

Optionally, the catalytic composition may further include water. When present, the water is preferably included in an amount up to about 50% v/v of the composition, more preferably up to about 10% v/v and most preferably about 1-5% v/v of the catalytic composition. When nitric acid is present in the catalytic composition, the amount of water is preferably about 2% v/v, while about 5% water is desirably included when nitric acid or a nitrate salt is included as the nitrogen-containing co-catalyst.

In a second embodiment, catalytic methods for oxidizing a reactant substrate are provided. The oxidation processes are preferably carried out by adding the reactant substrate to the catalyst composition to form a reaction medium, and pressurizing the reaction medium at constant volume with a gaseous oxygen source under conditions of temperature and constant pressure sufficient to selectively oxidize the reactant substrate to form a desired carboxylic acid product. The catalyst composition preferably includes the nitroxyl radical mediator, the nitrogen-containing co-catalyst and the organic acid. Preferably, the organic acid is a carboxylic acid that is different from a carboxylic acid produced from the oxidation of the reactant substrate in the reaction medium. A reactive substrate is preferably added to the catalytic composition and subsequently contacted with the oxygen source in the reaction vessel. The oxygen source is typically provided as oxygen gas or air at a desired pressure over the reaction media containing the reactive substrate within the reaction vessel. A fluid reaction medium comprising the substrate and catalytic composition may be charged in a jacketed glass reactor vessel connected to a volumetric manifold. The fluid reaction medium is preferably free of a bromide source. The reaction medium may be flushed multiple times with an oxygen-containing gas (preferably, oxygen gas or air) and heated to the target temperature (e.g., 65° C.). The oxygen-containing gas may be subsequently admitted to the targeted reaction pressure (e.g., about 45 psi) to initiate the oxidation reaction. The reaction media can be maintained at a suitable temperature during the oxidization reaction. The reaction media may be stirred to promote the aerobic catalytic oxidation of the reactive substrate in the reaction media.

In particular, the selective oxidation processes may selectively oxidize a primary alcohol pendant to a pyranose ring on a carbohydrate reactant substrate, under conditions of temperature and constant pressure sufficient to selectively oxidize the reactant substrate to form a second carboxylic acid, with the catalyst composition comprising the first carboxylic acid. The catalyst composition may optionally include a bromide source with the nitroxyl radical mediator and nitrogen-containing co-catalyst, although some aspects of the second embodiment provide for selective oxidation in the absence of a bromide source. The carbohydrate reactive substrate is preferably added to the catalytic composition and subsequently contacted with the oxygen source in the reaction vessel. The oxygen source is typically provided as oxygen gas or air at a desired pressure over the reaction media containing the reactive substrate within the reaction vessel. For pure oxygen, a constant pressure of about 1-300 psi, preferably about 5-50 psi, and most preferably about 45 psi. A fluid reaction medium comprising the substrate and catalytic composition may be charged in a jacketed glass reactor vessel connected to a volumetric manifold. The reaction medium may be flushed multiple times with an oxygen-containing gas (preferably, oxygen gas or air) and heated to the target temperature (e.g., 65° C.). The oxygen-containing gas may be subsequently admitted to the targeted reaction pressure (e.g., about 45 psi) to initiate the oxidation reaction. The reaction media can be maintained at a suitable temperature during the oxidization reaction, such as a temperature of between about 0° C. and about 100° C., but preferably above about 50° C. up to about 80° C., and most preferably about 65° C. The reaction media may be stirred to promote the aerobic catalytic oxidation of the reactive substrate in the reaction media.

Uptake of the oxygen-containing gas in the reaction vessel may be recorded against the time to monitor the progress of the oxidation reaction in the reaction media. The rate of oxygen uptake typically declines after oxidation of the reactive substrate, and the oxidized product can be subsequently isolated from the reaction media by any suitable method, such as rotary evaporation of the reaction media. The oxidation reaction is optionally performed in the absence of a bromide source, such as HBr or N-Bromosuccinimide (NBS). The product composition may be analyzed, for example, by HPLC using the first carboxylic acid, such as acetic acid (CH₃COOH), as an internal standard.

### BRIEF DESCRIPTION OF THE DRAWINGS

FIG. 1 is a schematic showing various chemical oxidation reaction schemes.

FIG. 2A is a chemical reaction scheme showing an oxidation product of ethylene glycol.

FIG. 2B is a Fischer projection reaction scheme showing an oxidation product of glucose.

FIG. 2C is a chemical reaction scheme showing an oxidation product of sucrose.

FIG. 3A is a chemical reaction scheme showing an oxidation product of methyl-α-d-glucopyranoside.

FIG. 3B is a chemical reaction scheme showing chemical reaction scheme of FIG. 3A as an intermediate reaction in obtaining methyl-α-d-glucuronic acid from starch.

FIG. 4 is a graph of oxygen consumption as a function of time during the oxidation of methyl glucoside to methyl glucopyranosiduronic acid using various amounts of magnesium nitrate in the reaction medium.

FIG. 5A is a graph of oxygen consumption as a function of time during the oxidation of methyl glucoside to methyl glucopyranosiduronic acid using various nitrogen-containing compounds (nitrate sources) in the reaction medium.

FIG. 5B is a graph of oxygen consumption as a function of time during the oxidation of methyl glucoside to methyl glucopyranosiduronic acid using various nitrogen-containing compounds (nitrate sources) in the reaction medium.

FIG. 6A is a graph of oxygen consumption as a function of time during the oxidation of methyl glucoside to methyl glucopyranosiduronic acid using different amounts of nitric acid in the reaction medium.

FIG. 6B is a graph of oxygen consumption as a function of time during the oxidation of methyl glucoside to methyl glucopyranosiduronic acid using different amounts of nitrate salt in the reaction medium.

FIG. 7 is a graph of oxygen consumption as a function of time during the oxidation of methyl glucoside to methyl glucopyranosiduronic acid using different amounts of AA-TEMPO in the reaction medium.

FIG. 8A is a graph of oxygen consumption as a function of time during the oxidation of methyl glucoside to methyl glucopyranosiduronic acid at different oxygen pressures and a first concentration of nitric acid.

FIG. 8B is a graph of oxygen consumption as a function of time during the oxidation of methyl glucoside to methyl glucopyranosiduronic acid at different oxygen pressures and a second concentration of nitric acid.

FIG. 9 is a graph of oxygen consumption as a function of time during the oxidation of methyl glucoside to methyl glucopyranosiduronic acid with a reaction medium comprising different amounts of nitric acid.

FIG. 10 shows two graphs of oxygen consumption as a function of time during the oxidation of methyl glucoside to methyl glucopyranosiduronic acid and two corresponding HPLC traces.

FIG. 11 is a calibration curve for the oxidation of methyl glucoside to methyl glucopyranosiduronic acid in acetic acid.

FIG. 12 is a graph of oxygen consumption as a function of time during the oxidation of methyl glucoside to methyl glucopyranosiduronic acid at different water concentrations in a reaction medium comprising nitric acid.

FIG. 13 is a graph of oxygen consumption as a function of time during the oxidation of methyl glucoside to methyl glucopyranosiduronic acid at different water concentrations in a reaction medium comprising sodium nitrate.

FIG. 14 is a graph of oxygen consumption as a function of time during the oxidation of methyl glucoside to methyl glucopyranosiduronic acid at different AA-TEMPO concentrations in a reaction medium comprising nitric acid.

FIG. 15 is a graph of oxygen consumption as a function of time during the oxidation of methyl glucoside to methyl glucopyranosiduronic acid at different sodium nitrate and AA-TEMPO concentrations in a reaction medium.

FIG. 16 is a graph of oxygen consumption as a function of time during the oxidation of methyl glucoside to methyl glucopyranosiduronic acid at different AA-TEMPO and nitric acid concentrations in a reaction medium.

FIG. 17 is a graph of oxygen consumption as a function of time during the oxidation of methyl glucoside to methyl glucopyranosiduronic acid using various amounts of nitric acid as a nitrate source in the reaction medium, with and without an NBS bromide source.

FIG. 18 is a graph of oxygen consumption as a function of time during the oxidation of methyl glucoside to methyl glucopyranosiduronic acid using various amounts of nitrogen dioxide as a nitrate source in the reaction medium, with and without an NBS bromide source.

FIG. 19 is a graph of oxygen consumption as a function of time during the oxidation of methyl glucoside to methyl glucopyranosiduronic acid using various amounts of nitric acid as a nitrate source in the reaction medium, with an NBS bromide source or an HBr bromide source.

### DETAILED DESCRIPTION

The present disclosure describes certain exemplary embodiments of catalytic compositions and methods for the oxidation of a reactive substrate. In a first embodiment, catalytic compositions suitable for oxidizing reactive substrates, such as sugar alcohols, are provided. In a second embodiment, methods for oxidizing the reactive substrates are provided that include bringing the substrate into reactive contact with the catalytic composition. Preferably, the oxidation reactions are performed in the absence of a bromide source.

### Definitions

As used herein, “derivative” refers to a chemically or biologically modified version of a chemical compound that is structurally similar to a parent compound and (actually or theoretically) derivable from that parent compound. A derivative may or may not have different chemical or physical properties of the parent compound. For example, the derivative may be more hydrophilic or it may have altered reactivity as compared to the parent compound. Derivatization (i.e., modification) may involve substitution of one or more moieties within the molecule (e.g., a change in functional group) that do not substantially alter the function of the molecule for a desired purpose. The term “derivative” is also used to describe all solvates, for example hydrates or adducts (e.g., adducts with alcohols), active metabolites, and salts of the parent compound. The type of salt that may be prepared depends on the nature of the moieties within the compound. For example, acidic groups, such as carboxylic acid groups, can form alkali metal salts or alkaline earth metal salts (e.g., sodium salts, potassium salts, magnesium salts and calcium salts, and also salts with quaternary ammonium ions and acid addition salts with ammonia and physiologically tolerable organic amines such as, for example, triethylamine, ethanolamine or tris-(2-hydroxyethyl)amine). Basic groups can form acid addition salts, for example with inorganic acids such as hydrochloric acid, sulfuric acid or phosphoric acid, or with organic carboxylic acids and sulfonic acids such as acetic acid, citric acid, benzoic acid, maleic acid, fumaric acid, tartaric acid, methanesulfonic acid or p-toluenesulfonic acid. Compounds which simultaneously contain a basic group and an acidic group, for example a carboxyl group in addition to basic nitrogen atoms, can be present as zwitterions. Salts can be obtained by customary methods known to those skilled in the art, for example by combining a compound with an inorganic or organic acid or base in a solvent or diluent, or from other salts by cation exchange or anion exchange.

As used herein, “analogue” or “analog” refers to a chemical compound that is structurally similar to another but differs slightly in composition (as in the replacement of one atom by an atom of a different element or in the presence of a particular functional group), but may or may not be derivable from the parent compound. A “derivative” differs from an “analogue” in that a parent compound may be the starting material to generate a “derivative,” whereas the parent compound may not necessarily be used as the starting material to generate an “analogue.”

Any concentration ranges, percentage range, or ratio range recited herein are to be understood to include concentrations, percentages or ratios of any integer within that range and fractions thereof, such as one tenth and one hundredth of an integer, unless otherwise indicated. Also, any number range recited herein relating to any physical feature, such as polymer subunits, size or thickness, are to be understood to include any integer within the recited range, unless otherwise indicated. It should be understood that the terms “a” and “an” as used above and elsewhere herein refer to “one or more” of the enumerated components. For example, “a” polymer refers to one polymer or a mixture comprising two or more polymers. As used herein, the term “about” refers to differences that are insubstantial for the relevant purpose or function.

The term “alcohols” as used herein include organic compounds having primary or secondary hydroxyl groups. Examples include alcohols such as methanol, ethanol, propyl alcohol, butyl alcohol, pentanol, 2-methyl-1-butanol, methyl-1-butanol, neopentyl alcohol, hexanol, 2-methyl-1-pentanol, neohexyl alcohol, heptanol, octanol, 2-ethyl-1-hexanol, nonyl alcohol, decyl alcohol, lauryl alcohol, dodecyl alcohol, eicosyl alcohol. Examples of unsaturated alcohols include allyl alcohol, crotyl alcohol and propargyl alcohol. Examples of aromatic alcohols include benzyl alcohol, phenyl ethanol, phenyl propanol and the like. Preferably, the reactant substrate is a sugar or sugar alcohol, such as a modified erythritol or xylitol structure, that includes one or more alcohol moieties pendant to a heterocyclic ring structure containing carbon and oxygen. For example, an alkyl-α-D-glucopyranoside (e.g., methyl-α-D-glucopyranoside, MGP) and other such modified sugar molecules with pendant primary alcohol moieties are particularly preferred substrates.

### Preferred Catalyst Compositions

In a first embodiment, catalytic compositions are provided. Preferably, the catalytic compositions include a nitroxyl radical, a nitrogen-containing co-catalyst and an organic acid. Table 3 discloses certain preferred catalyst compositions comprising various nitrate salts as nitrogen-containing co-catalyst, AA-TEMPO nitroxyl radical, acetic acid (“AcOH”) and water (“H₂O”). The table shows the milli-mole (mmol) quantities of the nitrogen-containing co-catalyst and AA-TEMPO nitroxyl radical in the catalyst composition, along with the volume of acetic acid and water in milliliters (mL). Similarly, Table 4 and Table 5 show preferred catalyst compositions containing sodium nitrate and/or nitric oxide as the nitrogen-containing co-catalyst (Table 4) or nitric acid as the nitrogen-containing co-catalyst (Table 5). Optionally, the catalytic compositions may further include a bromide source in combination with a nitroxyl radical, a nitrogen-containing co-catalyst and an organic acid.

The catalyst composition may comprise any suitable nitroxyl radical. The nitroxyl radical compound may be selected to possess a desired level of stability for an intended use. Preferably, the nitroxyl radical is a free nitroxyl radical mediator that is substantially stable at room temperature during storage for a minimum period of one week in the presence of oxygen. The nitroxyl radical is preferably a stable free nitroxyl radical which is not substituted by a hydrogen atom at any α-C-atom next to the nitrogen atom. Further, the “stable free nitroxyl radical” preferably retains a content of at least 90% of the free nitroxyl radical after storage for one week at 25° C. in the presence of oxygen, based on the initial content of free nitroxyl radical. For example, the free nitroxyl radical may be a compound described by the general formulae (I) or (II):

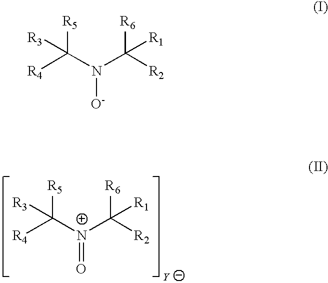

In formulae (I) and (II), R¹, R², R³, R⁴, R⁵, Ware independent of each other (C₁-C₁₀)-alkyl or alkenyl, (C₁-C₁₀)-alkoxy, (C₆-C₁₈)-aryl, (C₇-C₁₉)-aralkyl, (C₆-C₁₈)-aryl-(C₁-C₈)-alkyl or (C₃-C₁₈)-heteroaryl; R⁵ and R⁶ can also be bonded together via a (C₁-C₄)-alkyl chain, which can be unsaturated or substituted by one or more R¹, C₁-C₈-amido, halogen, oxy, hydroxy, amino, alkyl or dialkylamino, aryl or diarylamino, alkyl or arylcarbonyloxy and alkyl or arylcarbonylamino. In formula (II) the Y⁻ group is an anion.

Examples of free nitroxyl radicals or their oxoammonium derivatives include 2,2,6,6,-tetramethylpiperidine N-oxyl (TEMPO) and its 4-substituted derivatives such as 2,2,6,6-tetramethyl-4-methoxypiperidine-N-oxyl (MeO-TEMPO), 4-Methoxy-2,2,6,6-tetramethylpiperidine N-oxyl, 4-Hydroxy-2,2,6,6-tetramethylpiperidine N-oxyl (HO-TEMPO), 4-Benzoyloxy-2,2,6,6-tetramethylpiperidine N-oxyl (BnO-TEMPO), 4-Acetamino-2,2,6,6-tetramethylpiperidine N-oxyl (AA-TEMPO), N,N-Dimethylamino-2,2,6,6-tetramethyl-piperidine N-oxyl (NNDMA-TEMPO). The “heterogenized” forms of the nitroxyl radicals or their oxoammonium derivatives can also be used. As a solid support one can use inorganic supports, such as aluminum oxide, silica, titanium oxide or zirconium oxide or polymers, composites, carbon materials.

The nitroxyl radical is preferably 2,2,6,6,-tetramethylpiperidine N-oxyl (TEMPO) or an oxoammonium derivatives thereof, although suitable nitroxyl radical mediator may be used. Examples of other nitroxyl radical mediators include 4-substituted derivatives of TEMPO such as 4-Methoxy-2,2,6,6-tetramethylpiperidine N-oxyl, 4-Hydroxy-2,2,6,6-tetramethylpiperidine N-oxyl (HO-TEMPO), 4-Benzoyloxy-2,2,6,6-tetramethylpiperidine N-oxyl (BnO-TEMPO), 4-Acetamino-2,2,6,6-tetramethylpiperidine N-oxyl (AA-TEMPO), N,N-Dimethylamino-2,2,6,6-tetramethyl-piperidine N-oxyl (NNDMA-TEMPO). Preferably, the nitroxyl free radical mediator comprises 4-Acetamino-2,2,6,6-tetramethylpiperidine N-oxyl (AA-TEMPO).

The catalytic composition preferably also includes one or more different nitrogen-containing co-catalysts in combination with the nitroxyl radical mediator. Particularly preferred nitrogen-containing co-catalysts include one or more materials selected from the group consisting of: a nitrate source, nitrogen dioxide and nitric oxide. The nitrate source may include nitric acid, ammonium nitrate, alkyl ammonium nitrate or an alkali or alkaline-earth nitrate salt (e.g., magnesium nitrate). The nitrogen-containing co-catalyst may include combinations of these materials, such as nitrogen dioxide or nitric oxide in combination with nitric acid, or sodium nitrate or any alkali or alkaline-earth nitrate in combination with nitric acid. The nitrogen-containing co-catalyst composition is preferably present in the reaction media in a catalytically-effective amount. The nitrogen-containing co-catalyst composition is preferably soluble in an organic acid and is preferably non-toxic.

Optionally, some catalytic compositions include a trace amount of a bromide source. The bromide source is preferably included in an amount effective to function as a catalyst promoter, and is typically included in a trace amounts. For example, the amount of bromide source may be about 0.005-2.00 mol %, preferably about 0.1-2.0 mol %, preferably 0.05-0.50 mol %, with respect to the carbohydrate substrate (e.g., sugar or sugar alcohol). Any suitable bromide source may be used, including hydrobromic acid dissolved in acetic acid or any other bromide containing species, like HBr, NaBr, KBr, N-Bromosuccinimide (NBS), N-Bromophtalimide. When present, the bromide source is preferably NBS. Other compositions within the first embodiment are formulated without a bromide source.

The organic acid may be any acid that forms a homogeneous reaction mixture with the other components of the catalytic composition. Desirably, the liquid reaction media includes an organic acid that is a carboxylic acid different from an oxidation reaction product. The organic acid in the reaction media may be acetic acid, propionic acid or any other carboxylic acid that forms a homogeneous reaction mixture. Preferably, the carboxylic acid is acetic acid. The carboxylic acid may be used alone or in combination with other solvents. Optionally, additional solvents may be added to the reaction media to suspend the reactants. Preferred additional solvents may include acetonitrile, tetrahydrofuran, methylene chloride, ethyl acetate, acetone, chloroform, diethyl ether, methyl-tert butyl ether, dichloromethane and the like.

### Preferred Reactant Substrates

The catalytic composition may be formulated to be brought into reactive contact with one or more reactant substrates to perform catalytic chemical oxidation reactions described in the present disclosure. In particular, processes and catalytic compositions for the conversion of primary alcohols, secondary alcohols or aldehydes to carboxylic acids on various reactant substrates are provided.

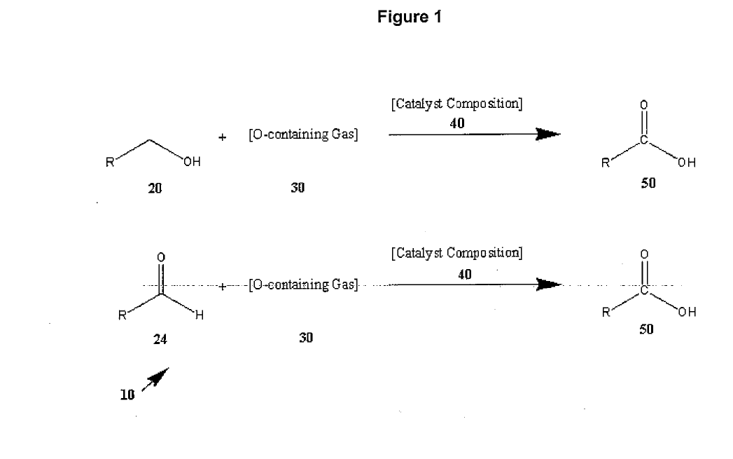

FIG. 1 is a schematic diagram certain oxidation reactants and products. Preferred processes 10 for the catalytic oxidation of primary and secondary alcohols are provided herein that comprise the steps of: (a) providing a first reactant 20 comprising a primary alcohol moiety, or a second reactant 24 comprising an aldehyde; (b) reacting the first reactant 20 or the second reactant 24 with an oxygen-containing gas 30 and in the presence of a catalyst composition 40 and (c) producing a first reaction product 50 comprising a carboxylic acid moiety from the first reactant 20 or the second reactant 24. The oxidation reactions illustrated in FIG. 1 are preferably performed in the absence of a bromide source such as an N-Bromosuccinimide (NBS) as a co-catalyst. The R moiety designates the remainder of a reactive substrate molecule, such as a heterocyclic group (e.g., a carbohydrate comprising a ring structure), an alcohol, or olefin moiety. R preferably includes 1 or more secondary alcohols that are not oxidized in the first reaction product 50.

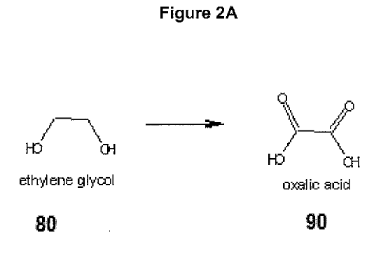

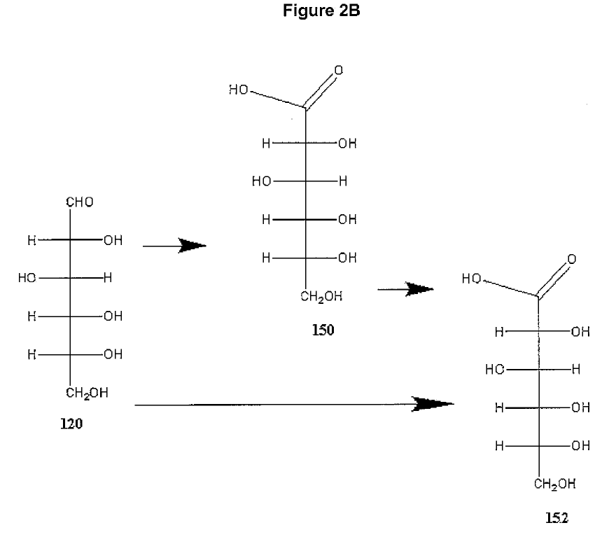

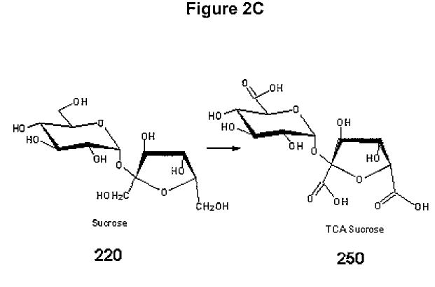

The selective oxidation is useful for the oxidation of any suitable reactant having a primary alcohol, a secondary alcohol or an aldehyde. The reactant may have any suitable structure comprising a primary alcohol. For example, the catalytic oxidation methods described herein can be used to oxidize a primary alcohol or polyol such as ethylene glycol 80 to the dicarboxylic acid oxalic acid 90, as shown in FIG. 2A.

Preferably, however, the reactant substrate is a cyclic carbohydrate having a pendant primary alcohol that is converted to a carboxylic acid by the selective oxidation process. In particular, methods for oxidizing a carbohydrate alcohol are provided, including the selective oxidation of primary alcohol groups joined to Cₙ carbohydrate rings at the n^{th}-Carbon position to carboxylic acids, where n is an integer equal to 3-6. Preferably, the reactant substrate comprises a carbohydrate ring structure that remains intact after the oxidation process. The carbohydrate reactant is preferably a pyranose or furanose ring having an primary alcohol or aldehyde that is oxidized during the oxidation reaction. The reactant may be one or more carbohydrates, such as a monosaccharide, disaccharide, oligosaccharide, polysaccharide, or any combination thereof. For example, the reactant may include a furanose ring, a cyclic hemiacetal of an aldopentose or a cyclic hemiketal of a ketohexose. In another example, the reactant may include a pyranose ring formed by the reaction of the C-5 alcohol group of a sugar with its C-1 aldehyde forming an intramolecular hemiacetal. Another type of suitable reactant includes two or more monosaccharides joined by a glycosidic bond. The carbohydrate reactant may include monosaccharides bonded via a dehydration reaction (also called a condensation reaction or dehydration synthesis) that leads to the loss of a molecule of water and formation of a glycosidic bond. The glycosidic bond can be formed between any hydroxyl group on the component monosaccharide. Specific examples of suitable reactant substrates include sucrose (table sugar, cane sugar, saccharose, or beet sugar) (i.e., glucose-fructose (α(1→2) sucrase)), lactose (milk sugar) (i.e., galactose-glucose (β(1→4) lactase)), maltose (i.e., glucose-glucose (α(1→4) maltase)), trehalose (i.e., glucose-glucose (α(1→1)α trehalase)), cellobiose (i.e., glucose-glucose (β(1→4) cellobiase)), maltose and cellobiose. Other reactive substrates may be disaccharides such as: gentiobiose (i.e., two glucose monomers with an β(1→6) linkage); isomaltose (i.e., two glucose monomers with an α(1→6) linkage); kojibiose (i.e., two glucose monomers with an α(1→2) linkage), laminaribiose (i.e., two glucose monomers with a β(1→3) linkage), mannobiose (i.e., two mannose monomers with either an α(1→2), α(1→3), α(1→4), or an α(1→6) linkage), melibiose (i.e., a glucose monomer and a galactose monomer with an α(1→6) linkage); nigerose (i.e., two glucose monomers with an α(1→3) linkage); rutinose (i.e., a rhamnose monomer and a glucose monomer with an α(1→6) linkage); and xylobiose (i.e., two xylopyranose monomers with a β(1→4) linkage).

The reactant is preferably a carbohydrate such as a sugar, sugar alcohol or derivative thereof having a ring structure or capable of forming a heterocyclic ring structure containing at least one oxygen. The reactant preferably comprises a pendant primary alcohol that does not form part of the ring structure, and that may be oxidized to form a carboxylic acid while preserving the substrate molecule's ring-forming capability. For example, the glucose molecule 120 shown in FIG. 2B may be used as a reactant. Preferred oxidation processes oxidize the C₆ primary alcohol moiety in glucose 120 to form an aldonic carboxylic acid (e.g., D-gluconic acid 150). Optionally, the C1 carbon may also be oxidized to a carboxylic acid to form glucaric acid 152, either directly from glucose 120 or from the aldonic acid 150. Optionally, additional oxidation may be performed at the C1 position to form an aldonic acid. The oxidative processes and catalyst compositions may oxidize alcohol moieties pendant to cyclic carbohydrate compounds while preserving the cyclic structure of the carbohydrate. Disaccharide reactants may also be oxidized using the methods described herein. For example, referring to FIG. 2C, sucrose 220 may be oxidized to TCA sucrose using the selective oxidation methods, preserving the dual ring structure of the reactant.

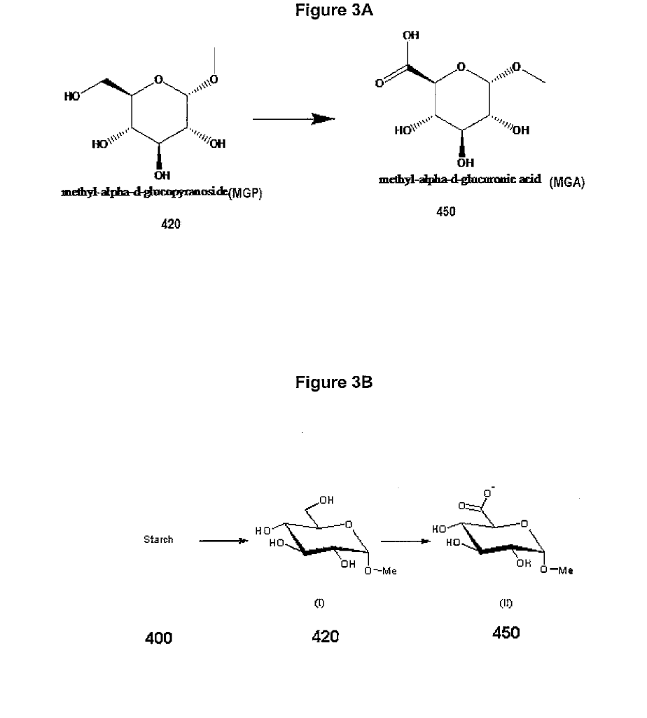

The selective oxidation methods and catalysts are discussed herein with respect to a preferred embodiment illustrated with reference to FIGS. 3A and 3B, describing the oxidation of an alkyl glucopyranoside such as methyl-α-D-glucopyranoside (MGP) 420 to a corresponding cyclic glucuronic acid, such as methyl-α-D-glucuronic acid 450 (MGA). Preferred processes for the catalytic oxidation of primary moieties in sugar alcohols are provided herein that comprise the steps of: (a) providing a reactant comprising a primary alcohol moiety; (b) reacting the reactant with an oxygen-containing gas and in the presence of a catalyst composition to produce (c) a reaction product where the primary alcohol moiety is converted to a carboxylic acid moiety. Preferred oxidation processes oxidize the C₆ primary alcohol moiety in glucose to form an aldonic carboxylic acid (e.g., D-gluconic acid). Optionally, the C₁ carbon may also be oxidized to a carboxylic acid to form glucaric acid, either directly from glucose or from the aldonic acid. Optionally, additional oxidation may be performed at the C₁ position to form an aldonic acid. The oxidative processes and catalyst compositions may oxidize alcohol moieties pendant to cyclic carbohydrate compounds while preserving the cyclic structure of the carbohydrate.

FIG. 3B shows a particularly preferred process wherein the MGP 420 is obtained from a starting material comprising starch 400. The MGP 420 may be obtained directly, or from any suitable starting material. Preferably, the starting material includes glucosyl group, a D-glucuronopyranosyl group or D-fructuronofuranosyl group. For example, the starting material may be a salt of D-glucuronic acid or a glycoside, oligomer, or polymer thereof, either as a natural material or produced by oxidation. Optionally, the starting material may be oxidized to a glucopyranosyl-containing moiety, or other oxidized products such as glucuronan, that can be converted to the desired product. Other suitable starting material compounds include glucosides, compounds with D-glucopyranosyl units in glycosidic linkages such as malto- or cellulo-oligo- or polysaccharides, or sucrose. The starting material preferably has either an alpha or beta configuration at carbon number 1. Alternatively, oligo- and polysaccharides containing 2,1-linked D-fructofuranosyl units may also serve as starting materials. In another aspect, a D-fructofuranosyl-containing compound or a 2,1-linked oligomer or polymer thereof produced by cleavage of a 2,1-linked fructan or oligomers obtained from it also may serve as the reactant substrate of the reaction sequence. Once obtained, the reactant comprising a primary and secondary alcohol moiety, such as the pendant primary hydroxyl group at the C₆ position of MGP 420 may be converted to MGA according to the catalytic oxidative processes disclosed herein.

Preferably, a carbohydrate reactant comprises a primary alcohol moiety positioned at the C-6 position of the glucopyranose ring. In one exemplary aspect, the reactant may comprise a carbohydrate having a glucopyranose ring. The carbohydrate reactant may also include one or more alkyl sugar derivatives, such as an alkyl-α-D-glucopyranoside. Methyl-α-D-glucopyranoside (MGP), a particularly preferred reactant. The methods permit oxidation of C₆ primary terminal hydroxyl groups in glucopyranose sugar alcohols, such as MGP, to carboxylic acids selectively without oxidizing secondary hydroxyl groups in the glucopyranose ring structure itself. Carbohydrates comprising two or more ring structures may also be used as substrates. For example, sucrose may be oxidized to TCA sucrose using the oxidation methods described herein. While the invention is discussed with reference to carbohydrate reactants, the oxidation methods disclosed are suitable for the oxidation of other primary alcohols or aldehyde groups. For example, the oxidation reaction of the first embodiment may be used to oxidize ethylene glycol to oxalic acid. Particularly preferred embodiments pertain to processes and catalysts for the oxidation of MGP to MGA. However, the oxidation methods and catalyst compositions are also applicable to other reactive substrates comprising primary or secondary alcohol moieties, such as alcohols or polyols (e.g., ethylene glycol) and sugars (such as glucose or fructose).

### Preferred Reaction Media

In a second embodiment, methods of oxidizing a reactant substrate are provided. The selective oxidation methods preferably comprise the steps of preparing a liquid reaction media comprising a catalyst composition described with respect to the first embodiment, combining the reactant substrate with the catalyst composition to form the reaction media, and pressurizing the reaction media at constant volume with a gaseous oxygen source under conditions of temperature and constant pressure sufficient to selectively oxidize a primary alcohol on the reactant substrate to a carboxylic acid. Preferably, the oxidation methods are performed in the absence of a transition metal catalyst or a hypochlorite reagent. Certain oxidation methods are performed with a bromide source, while other methods are performed without a bromide source.

The reaction media may be prepared by combining the catalyst composition with a suitable organic acid. Preferred reaction media are compositions of matter comprising: a reactant substrate, a nitroxyl radical, a nitrogen-containing co-catalyst, an organic acid and optionally containing a bromide source. Preferably, the reactant substrate is a sugar alcohol having at least one primary alcohol moiety pendant to a carbohydrate ring structure comprising a plurality of secondary alcohols. The organic acid is preferably a carboxylic acid.

Preferred reaction media are compositions of matter comprising: a reactant substrate, a nitroxyl radical, a nitrogen-containing co-catalyst, an organic acid, and optionally including a bromide source. Preferably, the reactant substrate is a sugar alcohol having at least one primary alcohol moiety pendant to a carbohydrate ring structure comprising a plurality of secondary alcohols. The organic acid in the catalyst composition is preferably a carboxylic acid such as acetic acid. The reaction media preferably includes the components of the catalytic composition and the reactive substrate in certain preferred molar ratios.

The reaction media preferably includes the components of the catalytic composition and the reactive substrate in certain preferred molar ratios. The amount of the nitrogen-containing co-catalyst in the reaction media may be optimized for a particular reaction to maximize the rate of oxidation (e.g., measured by the rate of oxygen consumption) and the selectivity for a desired oxidation product. Preferably, the reaction medium includes approximately equal molar amounts of the nitroxyl radical and a nitrogen containing co-catalyst (i.e., a molar ratio of about 1.0). In another aspect, the reaction medium includes the nitroxyl radical and the nitrogen containing co-catalyst in a molar ratio of between about 0.2 (i.e., about 1:5) and 5.00, including ratios of about 0.27, 0.40, 0.50, 0.80, 0.82, 1.00, 1.25, 1.67 and 2.50.

The ratio of the nitroxyl radical to reactive substrate in the reaction media may be varied depending on the particular reactant, the reaction conditions and the other components of the reaction media. Preferably, this ratio is minimized. For example, the nitroxyl radical may be present in the reaction media an amount of from about 0.001-10 mol %, based on the amount of said reactive substrate. Preferably, the catalytic amount of nitroxyl radical is 0.1-5.0 mol %, 0.1-3.5 mol %, or most preferably 0.1-1.0 mol %, with respect to the reactive substrate. The amount of stable free nitroxyl radical includes all values and subvalues therebetween, especially including 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.5, 3, 3.5, 4, 4.5, 5, 5.5, 6, 6.5, 7, 7.5, 8, 8.5, 9, and 9.5 mol % of the reactive substrate in the reaction media. For example, the reaction medium may include about 0.33 mol % to about 3.33 mol % of the nitroxyl radical compared to the reactive substrate. Preferably, the catalytic amount of nitroxyl radical is 0.1-2 mol %, and most preferably 0.5-1 mol % with respect to the reactive substrate in the reaction media. In another aspect, the molar ratio of the reactive substrate to the nitroxyl radical mediator in the reaction media is at least about 1:1, more preferably about 10:1 and most preferably about 10:1 to about 500:1, most preferably about 30:1 to about 300:1, or greater. Examples of suitable molar ratios between the reactive substrate and the nitroxyl radical include 30:1, 38:1, 44:1, 50:1, 75:1, 80:1, 75:1, 80:1, 100:1, 150:1 and 300:1. In one aspect, the molar ratio of the reactive substrate to the nitroxyl radical is about 1.0-200 (i.e., 1:1-200:1), including ratios of about 1 (e.g., 1.83), 3, 4, 20, 30, 75, and 188, and more preferably about 1.0-100.

The amount of the nitrogen-containing co-catalyst in the reaction medium may be 0.1-10.0 mol %, preferably 0.30-5.0 mol % with respect to the reactive substrate. Examples of suitable amounts of the nitrogen-containing co-catalysts include about 0.1-2.0 mol %, preferably about 0.50-0.75 mol %, of the co-catalyst with respect to the reactive substrate (e.g., a sugar alcohol). Preferably, the reaction medium includes approximately equal molar amounts of free nitroxyl radical (i.e., a molar ratio of about 1.0). The optimal amount of the nitrogen-containing co-catalyst composition in the reaction may be optimized for a particular reaction to maximize the rate of oxidation (e.g., measured by the rate of oxygen consumption) and the selectivity for a desired oxidation product.

When a bromide source is present in the reaction media, the molar ratio of the reactive substrate to the bromide source is about 30 or greater (i.e., 30:1), and typically about 20-600, including ratios of about 37.5, 120, 150, 187, 200, 300, 600, 882 and 1667. Preferably, the molar ratio of the nitroxyl radical to the bromide source is about 1.0-100, and typically about 1.0-60, including ratios of about 1.0-30, 1.0-30, 1.0-10, 1.5, 2.5, 3.2, 4.0, 5.0, and 6.67.

The reaction media may optionally include a suitable amount of water. In some embodiments, the reaction media includes water as co-solvent, leading to an increase in the rate of desired oxidation reactions. For example, water may be present in the reaction media amounts ranging from 0.1-1000 mol %, preferably 50-100 mol % with respect to the reactive substrate. In one aspect, the liquid reaction medium is an aqueous solution comprising up to about 50% v/v water. In another aspect, the liquid reaction media includes about 1%-10% v/v water.

One preferred reaction media includes water and a nitrate source in amounts that maximize the oxidation rate of the substrate at a desired level of selectivity for the reaction. The ratio of nitroxyl catalyst to a sugar alcohol substrate is preferably kept as low as possible. For example, the concentration of nitric acid as a nitrogen-containing co-catalyst is preferably about 0.1 to 10.0 mol %, more preferably about 0.1-7.0 mol %. In one aspect, the amount of nitroxyl radical in the reaction media may be 0.17-6.67 mol %, including amounts of about 0.17, 0.33, 0.67, 1.33, 1.67, 2.00, 2.25, 2.67, 3.33, 4.00, 4.67, 5.00, 5.33, 6.00, 6.50 and 6.67 mol % with respect to the reactive substrate. In another aspect, the molar ratio of the substrate to the nitrogen-containing co-catalyst in the reaction media is at least about 10:1, more preferably about 50:1 and most preferably about 100:1, 200:1, 300:1, 600:1 or greater. Molar ratios of the substrate to the nitrogen-containing co-catalyst are typically between about 10:1 to 600:1 (or greater), but are preferably maximized. Examples of suitable molar ratios of the substrate to the nitrogen-containing co-catalyst include 15.00, 15.38, 16.67, 18.75, 20.00, 21.43, 25.00, 30.00, 37.50, 40.00, 44.40, 50.00, 60.00, 75.00, 150.00, 300.00, and 600.00 to one. Particularly preferred ratios of the substrate to the nitrogen-containing co-catalyst are about 100:1 or greater, including 100:1 to about 600:1.

Another preferred reaction media includes water, a bromide source and a nitrate source in amounts that maximize the oxidation rate of the substrate at a desired level of selectivity for the reaction. For example, the concentration of nitric acid as a nitrogen-containing co-catalyst is preferably about 0.66 to 6.6 mol %, more preferably about 2.7-3.3 mol %. The ratio of nitroxyl catalyst to a sugar alcohol substrate is preferably kept as low as possible. In one aspect, the amount of nitroxyl radical in the reaction media may be 0.1-2 mol %, and most preferably 0.5-1 mol % with respect to the reactive substrate. In another aspect, the molar ratio of the substrate to the nitroxyl radical mediator in the reaction media is at least about 40:1, more preferably about 100:1 and most preferably about 120:1, 150:1, 190:1, or greater.

Preferably, the reaction medium includes relative molar amounts of the nitroxyl radical and the nitrogen-containing co-catalyst that provide a desired reaction rate and conversion percentage. Typically, the molar ratio between the nitroxyl radical and the nitrogen-containing co-catalyst is between about 1:10 and 100:1, preferably about 1:10 to about 50:1, and most preferably between about 1:10 and 20:1. Examples of suitable molar ratios with less of the nitroxyl radical than the nitrogen-containing co-catalyst, including molar ratios of 1:x where x is 8.00, 7.50, 7.50, 6.50, 5.03, 5.00, 4.50, 4.00, 3.75, 2.50, 2.00, 1.80, 1.67, 1.60, 1.40, 1.33, or 1.20. Examples of suitable molar ratios with less of the nitroxyl radical than the nitrogen-containing co-catalyst, including molar ratios of y:1 where y is 1.00, 1.25, 1.50, 1.67, 2.00, 2.50, 3.00, 5.00, 10.00 or 20.00.

Preferably the amount of the organic acid (e.g., a carboxylic acid such as acetic acid) in the reaction medium is about 0.1-200 mol %, preferably 0.5-50 mol %, more preferably about 0.5-5.0 mol % and most preferably about 0.5-1.5 mol % with respect to a reactive substrate, including 0.86%, 0.95%, 1.17%, 1.19%, 1.31%, 1.32% and 1.34% mol %. By volume, the reaction solution and/or catalyst composition may contain any suitable amount of the organic acid that provides a desired reaction rate and selectivity of the oxidation reaction. Typically, the catalytic composition contains the nitroxyl radical and the nitrogen-containing co-catalyst in an organic acid, where the organic acid constitutes about 10-100% of the liquid volume (v/v) of the reaction medium. The reaction medium further comprises the reactive substrate dissolved or suspended in the organic acid. Preferably, the liquid volume of the reaction medium includes about 50-100%, more preferably about 85-100%, and most preferably about 94%, 95%, 96%, 97%, 98%, 99% or 100% of the organic acid.

The reaction media may optionally include a suitable amount of water. Typically, when water is present in the reaction medium, the liquid volume of a reactant medium consists of the organic mixture and the water. In some aspects, the water acts as a co-solvent in the reaction medium, leading to an increase in the rate of desired oxidation reactions. For example, water may be present in the reaction media amounts ranging from 0.1-1000 mol %, preferably 50-100 mol % with respect to the reactive substrate. In one aspect, the liquid reaction medium is an aqueous solution comprising up to about 50% v/v water. In another aspect, the liquid reaction media includes about 1%-10% v/v water. Examples of suitable amounts of water in the reaction medium include: 0.00%, 0.91%, 1.74%, 2.13%, 2.17%, 2.38%, 4.96%, 5.22% and 49.55%.

Examples of particularly preferred reaction media compositions are listed in Table 3, Table 4 and Table 5 below. Each table (Tables 3, 4 and 5) discloses certain preferred reaction media comprising various catalyst compositions in combination with an MGP reactive substrate. Each table also includes certain ratios within the preferred reaction media compositions: the molar ratio (“MGP:TEMPO”) of the MGP reactive substrate (MGP) to nitroxyl radical (AA TEMPO), the molar ratio (“MGP:NCCC”) of the MGP reactive substrate (MGP) to the nitrogen-containing co-catalyst (NCCC), the molar ratio (“MGP:(TEMPO+NCCC)”) of the MGP reactive substrate to the total moles of the nitroxyl radical (AA TEMPO) and the nitrogen-containing co-catalyst (NCCC), the percentage by volume of water in the reaction media (“H2O %”) and the percent by volume of the acetic acid (AcOH) organic acid (“AcOH %”).

### Preferred Catalytic Oxidation Processes

The selective oxidation methods may be performed by contacting a reactant substrate comprising a primary alcohol moiety with an oxygen source in a liquid reaction medium containing a catalyst composition under conditions of temperature and pressure effective to oxidize the alcohol moiety to a carboxylic acid. Preferably, the process uses molecular oxygen or air as an oxidant and may be performed at desirably high substrate to catalyst molar ratios. A reaction media and reactive substrate may be charged in a jacketed glass reactor vessel connected to a volumetric manifold. The reaction mixture may be flushed multiple times with an oxygen-containing gas (preferably, oxygen gas or air) and heated to the target temperature (e.g., 65 C).

An oxygen-containing gas may subsequently be admitted to a suitable reaction pressure to initiate the oxidation reaction. Preferably, the oxygen pressure is selected to minimize the required oxygen pressure required to provide a desired reaction rate. The oxygen containing gas may be pure oxygen, air, or a mixture of oxygen and an inert gas or air. The oxygen partial pressure may be selected depending on the reactive substrate but is not particularly limited. The oxygen source is typically provided as oxygen gas or air at a desired pressure over the reaction media containing the reactive substrate within the reaction vessel, typically at a constant pressure of about 5-200 psi, preferably about 10-50 psi, and most preferably about 45 psi. When pure oxygen is used, the reaction pressure is in the range 2-200 psi, preferably 10-50 psi. Using air, the reaction pressure is in the range of 1-900 bar, preferably 30-250 psi.

The reaction media can be maintained at a suitable temperature during the oxidization reaction, such as a temperature of between about 0° C. and about 100° C., but preferably about 20° C. to about 80° C. and most preferably about 65° C. The reaction temperature at which the process of the invention is carried out depends on the reactivity of the alcohol substrate and is in general between 0° C. and 100° C., preferably between 20° C. to 80° C., most preferably between 40° C. to 50° C. The reaction temperature may be selected depending on the reactivity of the reactive substrate. The reaction temperature may also be 10, 20, 30, 40, 50, 60, 70, 80 and 90° C., or any temperature therebetween.

The reaction media may be stirred to promote the aerobic catalytic oxidation of the reactive substrate in the reaction media. Uptake of the oxygen-containing gas in the reaction vessel may be recorded against the time to monitor the progress of the oxidation reaction in the reaction media. The rate of oxygen uptake typically declines after oxidation of the reactive substrate.

For example, an oxidation method preferably comprises the steps of: (1) preparing a reaction media by combining a catalyst composition containing a free nitroxyl radical and a nitrate source co-catalyst with acetic acid to form a reaction medium, (2) adding a reactive substrate comprising an alkyl glucopyranoside carbohydrate to form a suspension in the reaction medium, (3) heating the reaction medium suspension to a desired temperature in a reaction vessel while stirring under a low oxygen pressure in the reaction vessel, (4) pressurizing the reaction vessel with an oxygen-containing gas to a suitable reaction pressure, (5) permitting the oxidation reaction to occur in the reaction medium suspension, (6) monitoring the consumption of oxygen-containing gas in the reaction vessel as the reaction proceeds, while maintaining a constant pressure of oxygen-containing gas in the reaction vessel, (7) cooling the reaction vessel and reaction medium after the consumption of oxygen-containing gas by the reaction subsides, and (8) removing the acetic acid from the reaction medium and isolating the oxidized reaction product.

The oxidation processes of the present invention can be run as a batch, a semi-batch or as a continuous process, and may be performed in any suitable reactor type or configuration. Thus, stirred tank reactors, tube reactors, reactor cascades, micro reactors or any possible combination of those reactor types might be used.

The oxidation product can be worked up by any known methods such as by phase-splitting, removal of the solvent by distillation, ion exchange or membrane separation techniques. Depending on the requirements for purity, any other chemical methods can also be used. For example, the oxidized product can be subsequently isolated from the reaction media by any suitable method, such as rotary evaporation of the reaction media. For example, the product composition may be analyzed by HPLC using acetic acid (CH₃COOH) as an internal standard.

A series of liquid phase aerobic oxidations of MGP in a reaction medium containing acetic acid were carried out in the presence of a catalyst composition comprising a AA-TEMPO nitroxyl radical and a nitrogen-containing co-catalyst in the absence of a bromide source (e.g., NBS or HBr). The nitrogen-containing co-catalyst contained one or more compounds selected from the group consisting of: nitrate sources, nitric acid, nitric oxide and nitrogen dioxide.

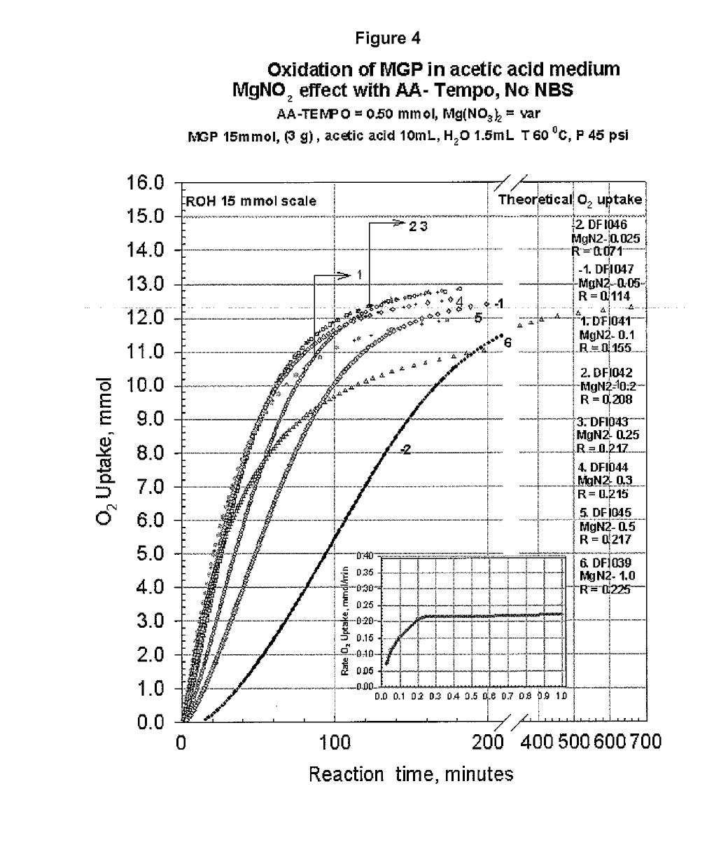

In a first experiment, varying concentrations of magnesium nitrate (Mg(NO₃)₂) were added to a reaction medium consisting essentially of: 0.5 mmol AA-TEMPO, 15 mmol of the MGP substrate (3 g), 10 mL acetic acid and 1.5 mL water. The oxidation of MGP was performed at 60 C and 45 psi. oxygen pressure in the reaction vessel. The Mg(NO₃)₂ concentration was varied to determine effect of the [AA-TEMPO]:[NO₃⁻] ratio on selectivity and reaction rate for MGP oxidation. The conversion and the selectivity data are listed in Table 1 below and the oxygen uptake curves are plotted in FIG. 4.

TABLE 1

| | | Rate O₂ | | |
|---|---|---|---|---|
| Curve | mmol | consumption | | |
| (FIG. 4) | Mg(NO₃)₂ | (mmol/min) | Conversion % | Selectivity % |
| −1 | 0.050 | 0.1114 | 100 | 100 |
| −2 | 0.025 | 0.071 | 100 | 100 |
| 1 | 0.100 | 0.155 | 100 | 100 |
| 2 | 0.200 | 0.208 | 100 | 100 |
| 3 | 0.250 | 0.217 | 100 | 100 |
| 4 | 0.300 | 0.215 | 100 | 100 |
| 5 | 0.500 | 0.217 | 100 | 100 |
| 6 | 0.500 | 0.117 | 100 | 100 |

The inset in FIG. 4 shows the effect of the NO₃⁻ concentration on the rate of oxygen uptake. Preferably, the molar ratio of [(NO₃)⁻]:[AA-TEMPO] is about 1.0, which may correspond to a maximum rate of oxidation of about 2.17 mmol/min.

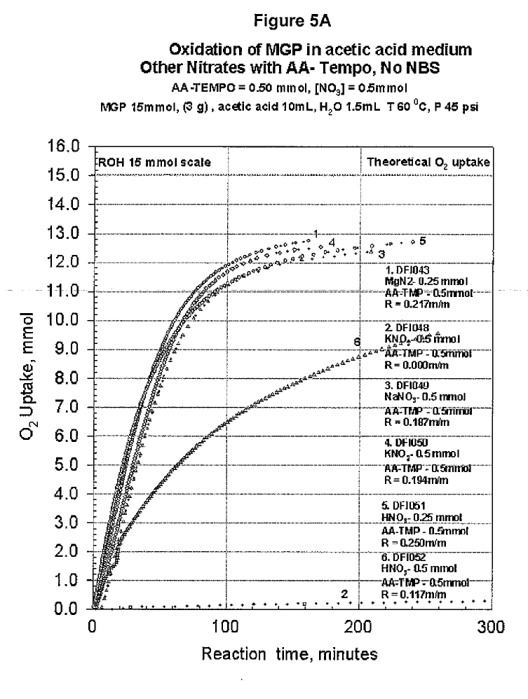

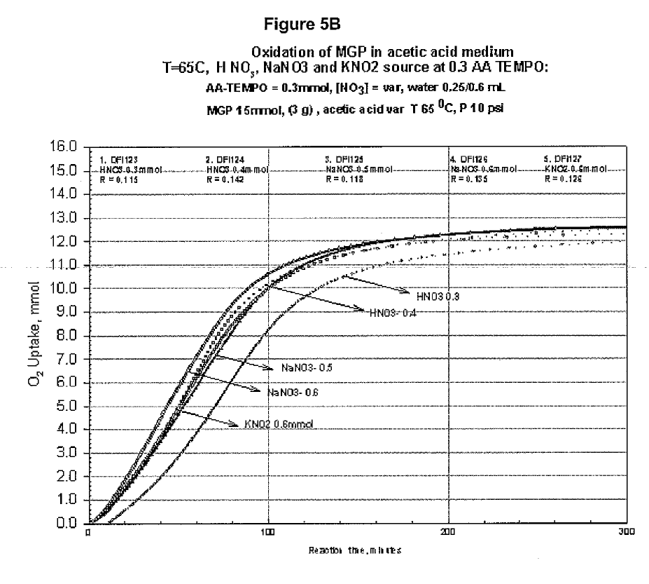

Several other nitrogen-containing compound nitrate sources were also tested in the liquid phase aerobic oxidations of MGP similar to the reactions shown in FIG. 4. FIG. 5A shows a series of oxygen uptake curves 110 for the oxidation of MGP in a reaction medium containing acetic acid carried out in the presence of a catalyst composition comprising AA-TEMPO and a variety of other nitrate (NO₃⁻) salts. The oxygen uptake curves in FIGS. 5A-5B were obtained from reactions that were the same as the reactions depicted in oxygen uptake curves in FIG. 4, except that different nitrate salts were used. The reaction medium containing 0.5 mmol AA-TEMPO, 15 mmol of the MGP substrate (3 g), 10 mL acetic acid and 1.5 mL water. The oxidation of MGP was performed at 60 C and 45 psi. oxygen pressure in the reaction vessel.

In a second aspect, the nitrogen-containing co-catalyst comprises nitric acid (HNO₃). FIG. 5A shows the oxygen uptake curves for a bromide-free reaction media consisting essentially of: 0.5 mmol Mg(NO₃)₂, 0.5 mmol KNO₃, 0.5 mmol NaNO₃, 0.5 mmol KNO₂, 0.25 mmol HNO₃ and 0.50 mmol HNO₃. Notably, higher reaction rates were observed when the HNO₃ concentration was reduced to 0.25 mmol (i.e., the molar ratio of [(NO₃)⁻]:[AA-TEMPO] is about 2.0), with the HNO₃ based system comparable to the most active MgNO₃ and NaNO₃ based counterparts. Accordingly, a reaction medium comprising nitric acid preferably includes the nitroxyl radical mediator and nitric acid in a molar ratio of about 2:1. FIG. 5B shows the oxygen uptake curves for a bromide-free reaction media consisting essentially of the compositions listed in Table 2 at 65° C.

TABLE 2

| | | Amount of | | | Rate of | | |
|---|---|---|---|---|---|---|---|
| | | nitrate | | | oxygen | | |
| | Nitrate | source | AA-TEMPO | | consumption | % | |
| Curve | source | (mmol) | (mmol) | Water (mL) | (mmol/min) | Conversion | Selectivity |
| 1 | HNO₃ | 0.3 | 0.3 | 0.25 | 0.115 | 100 | 100 |
| 2 | HNO₃ | 0.4 | 0.3 | 0.25 | 0.142 | 100 | 100 |
| 3 | NaNO₃ | 0.5 | 0.3 | 0.60 | 0.118 | 100 | 100 |
| 4 | NaNO₃ | 0.6 | 0.3 | 0.60 | 0.135 | 100 | 100 |
| 5 | KNO₂ | 0.6 | 0.3 | 0.60 | 0.126 | 100 | 100 |

The data in FIG. 5B and Table 2 suggest that at 65° C., the HNO₃ and NaNO₃ co-catalysts are similar in performance. The full conversion of the MGP was achieved over 180 min reaction time and the calculated productivity of the system at conditions is 16 mol MGA/mol catalyst/h, (assuming the catalyst is considered the AA-TEMPO component).

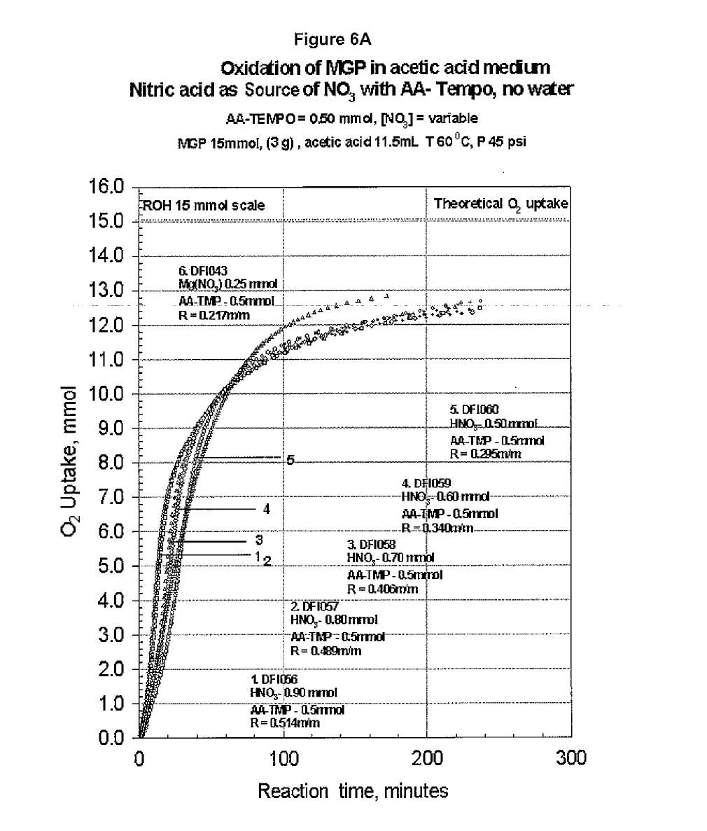

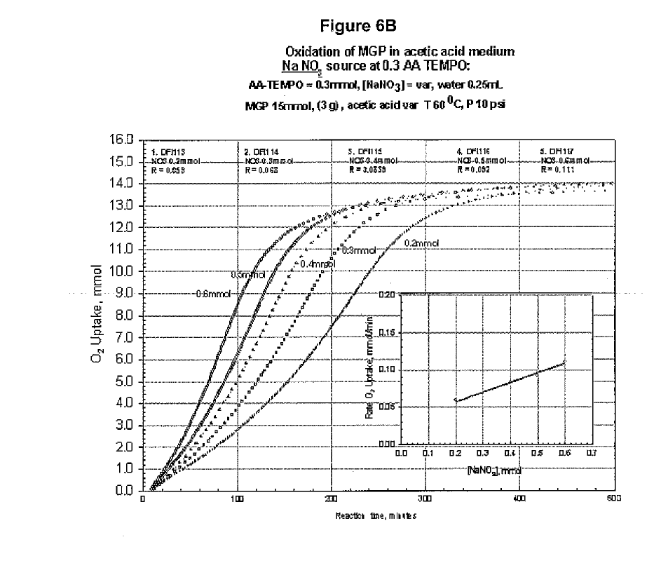

Other experiments were performed using a reaction media comprising nitric acid and water in various amounts. The amount of water is preferably selected to maximize the oxidation rate of the substrate at a desired level of selectivity for the reaction. The concentration of nitric acid is preferably about 0.66 to 6.6 mol %, more preferably about 2.7-3.3 mol %. FIG. 6A shows the oxygen uptake curves for liquid phase oxidation reactions of MGP conducted in a pressure reactor with varied concentrations the nitric acid from 0.1 mmol (0.66 mol %)-1 mmol (6.6 mol %) HNO₃ in a reaction medium consisting essentially of 0.5 mmol AA-TEMPO, 15 mmol (3 g) MGP, and 11.5 mL dry acetic acid at 60° C. and an oxygen pressure of 45 psi, resulting in an initial increase in the rate of oxygen uptake without substantially affecting the 200-minute full reaction time for full oxidation of the MGP (data not shown). A first order relationship was observed between the rate of oxidation and the HNO₃ concentration at low concentrations. However, at a reaction medium nitric acid concentration of 0.4-0.5 mmol (2.7-3.3 mol %), the total reaction time became independent from the further increase in the nitrate concentration. Additional MGP oxidation reactions carried out by varying the water content of a reaction medium consisting essentially of 0.5 mmol nitric acid, 0.5 mmol AA-TEMPO, 15 mmol (3 g) MGP, and 11.5 mL dry acetic acid with varying amounts water content in the acetic acid from 0-1 mL resulted in a maximum oxidation rate at 0.25 mL (2.1% v/v water in the reaction medium). Accordingly, a reaction medium comprising nitric acid also preferably includes about 2.1% v/v water.

FIG. 6B shows the oxygen uptake curves for liquid phase oxidation reactions of MGP conducted in a pressure reactor with varied concentrations the sodium nitrate (NaNO₃) from 0.2 mmol-0.6 mmol NaNO₃ in a reaction medium consisting essentially of 0.3 mmol AA-TEMPO, 15 mmol (3 g) MGP, and 11.25 mL acetic acid with 0.25 mL water at 60° C. and an oxygen pressure of 10 psi. The rate of oxygen uptake was first order with respect to the NaNO₃ concentration in the 0.2-0.6 mmol range (a linear relationship was observed between the oxidation rate and the NO₃ concentration in the liquid phase). Comparable rates were recorded when the same five concentrations of NaNO₃ were tested at higher water content of 0.6 mL (5.2% v/v).

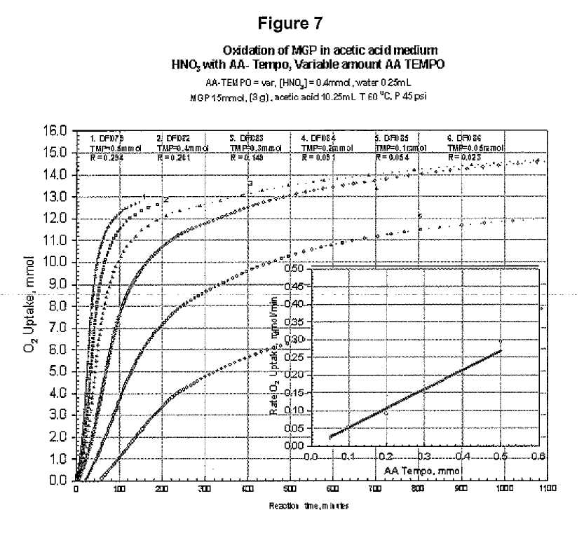

FIG. 7 shows oxygen uptake curves for MGP oxidation reactions carried out by varying the AA-TEMPO content of a reaction medium consisting essentially of 0.4 mmol nitric acid, 15 mmol (3 g) MGP, and 10.25 mL acetic acid with 0.25 mL water (2.1% v/v) with varying amounts AA-TEMPO content from 0.05-0.5 mmol. The data shown in FIG. 7 suggests that the initial rate of oxygen uptake is of first order to the AA-TEMPO concentration but at higher concentrations, the time for complete conversion shows tendency of leveling off. The productivity of the system was calculated as 12 mol MGA/mol catalyst/h where the catalyst is considered the AA-TEMPO component.

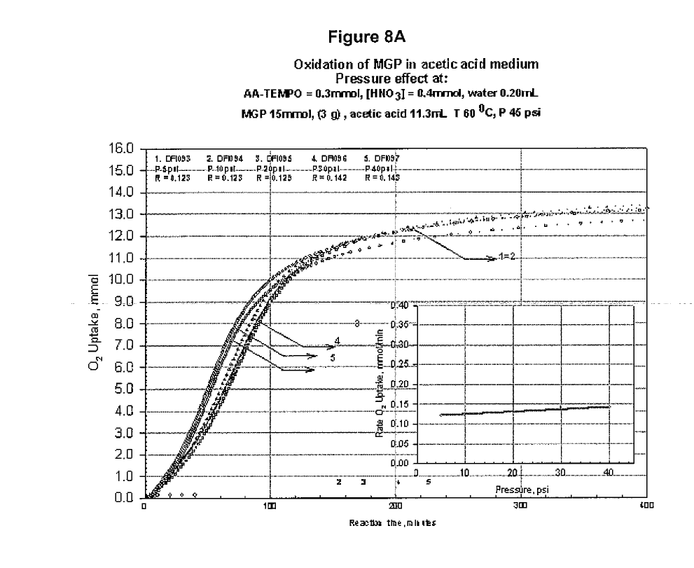

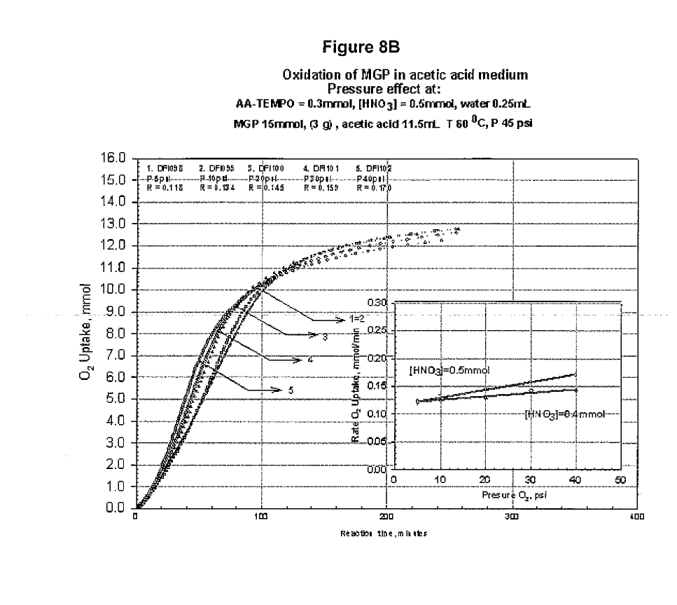

FIG. 8A shows oxygen uptake curves for MGP oxidation reactions carried out by varying the oxygen pressure in the reactor of a reaction medium consisting essentially of 0.3 mmol AA-TEMPO (2 mol %), 0.4 mmol nitric acid, 15 mmol (3 g) MGP, and 11.3 mL acetic acid with 0.2 mL water at 60° C. with varying oxygen pressures from 5-40 psi; FIG. 8B shows oxygen uptake curves for MGP oxidation reactions carried out by varying the oxygen pressure in the reactor of a reaction medium consisting essentially of 0.3 mmol AA-TEMPO (2 mol %), 0.5 mmol nitric acid, 15 mmol (3 g) MGP, and 11.5 mL acetic acid with 0.25 mL water at 60° C. with varying oxygen pressures from 5-40 psi. The data shown in FIG. 8A suggests that at low concentrations of nitric acid of 0.4 mmol, the oxygen pressure has a relatively little effect on the reaction rate (see the inset of FIG. 8A). The data shown in FIG. 8B suggests that increasing the concentration of the nitric acid to 0.5 mmol (3.3 mol %) appears to make the reaction more sensitive to the oxygen pressure and a pressure increase from 5 psi to 40 psi leads to a 30% increase in the rate of oxygen uptake.

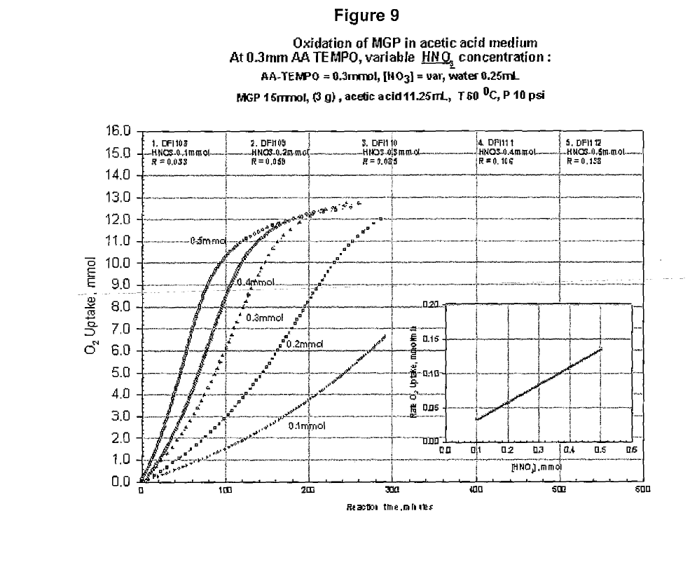

Oxygen uptake curves for certain particularly preferred reaction media are shown in FIG. 9. FIG. 9 shows oxygen uptake curves for MGP oxidation reactions carried out by varying the oxygen pressure in the reactor of a reaction medium consisting essentially of 0.3 mmol AA-TEMPO (2 mol %), 15 mmol (3 g) MGP, and 11.25 mL acetic acid with 0.25 mL water at 60° C. and 10 psi oxygen pressure, with varying nitric acid amounts from 0.1-0.5 mmol. Preferably, a reaction medium comprising nitric acid also comprises the MGP substrate and about 2 mol % [AA-TEMPO] (0.3 mmol), and a nitric acid concentration of about 3.3 mol % (0.5 mmol) or higher at a lower oxygen pressure of about 10 psi, permitting productivity in the range of 12 mol MGA/mol catalyst/h.

The process of this invention will be further described by the following examples, which are provided for illustration and are not to be construed as limiting the invention.

### EXAMPLES

The following description of the reagents, reactor configuration, and experimental methods shall apply to each exemplary methyl-α-D-glucopyranoside (MGP) oxidation reaction, unless otherwise stated. An acetamido TEMPO catalyst and the Methyl-α-D-glucopyranoside (MGP) were purchased from Aldrich. In the following examples, the oxidation reactions were performed using an in-house made Multi Autoclave Glass Volumetric system. All reactions were carried out in Ace Glass reaction flasks with Teflon heads, equipped with Swagelock based injection and thermocouple ports. Digital stirrers and Fisher cross stir bars were used for providing an efficient stirring. The Multi Autoclave reactor system allows conducting five simultaneous oxidations with five independent variables. The reactor system permits the recording of the oxygen uptake with the time, thus allowing precise monitoring the progress of the oxidation.

### Examples 1-12

Examples 1-12 and Table 3, Table 4 and Table 5 disclose certain preferred reaction media and the reaction data for the selective oxidation of MGP to MGA. Each MGP oxidation reaction in Table 3, Table 4 and Table 5 was carried out as follows, unless otherwise stated. Unless otherwise stated, these reaction compositions were formulated without a bromide source.

The reactor medium comprising MGP, Mg(NO₃)₂, AA-TEMPO, CH₃COOH and/or water was maintained without a bromide source throughout the reaction. The reactor medium was loaded in the reactor and the flask connected to a volumetric manifold. The flask was flushed three times with oxygen and immersed in the thermostated water bath held at 60° C. The oxygen pressure was brought to the required level using the by-pass line and the oxygen admitted at the total process pressure. The continuous monitoring of the oxygen uptake was initiated and recorded against the time. After the reaction was completed, the product composition was analyzed by HPLC using the reaction solvent (CH₃COOH) as an internal standard. Unless otherwise stated, all reactions in this section were run at the following standard conditions: 15 mmol scale (2.97 g MGP), T=60° C., P=45 psi, total solvent volume 11.5 mL at the ratio CH₃COOH (acetic acid):H₂O=10:1.5.

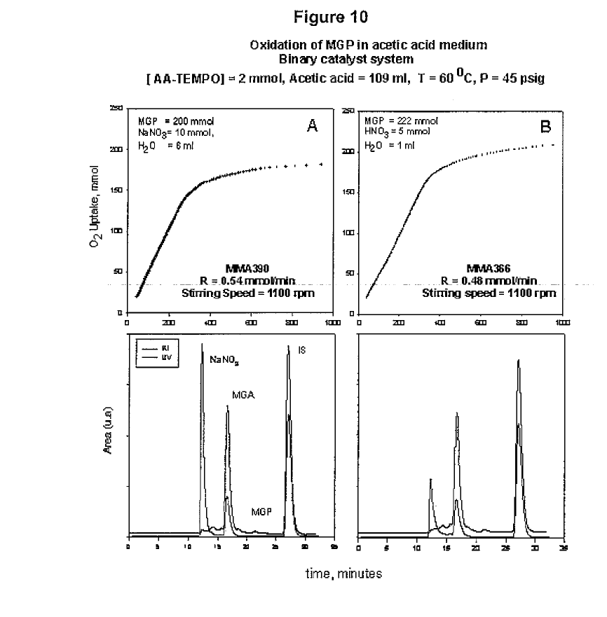

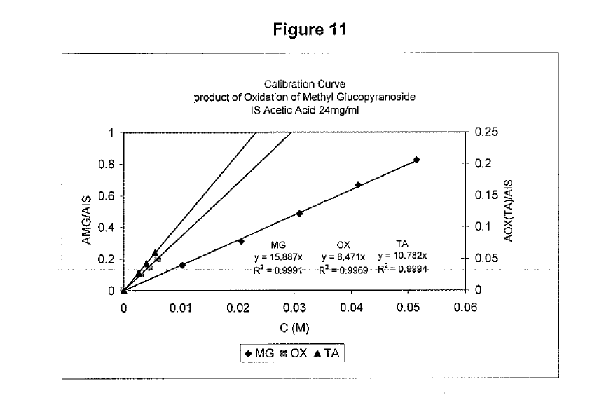

In a typical MGP oxidation reaction using sodium nitrate (NaNO₃), the flask is charged with MGP (38.7 g, 199.3 mmol), AA-TEMPO (0.427 g, 2.0 mmol), NaNO₃ (0.84 g, 10 mmol), acetic acid (CH₃COOH; 109 mL, 114.45 g, 1.90 mol) and H₂O (6 g, 333 mmol). The flask was purged three times with oxygen, pressurized initially to 45 psi and the circulation of the heating liquid was initiated. When the temperature of the reaction mixture reached 60° C., the pressure was adjusted to 45 psi and the computer monitoring of the oxygen uptake started. After the consumption of 152 mmol of oxygen, which at these conditions is completed in 13.3 hours, the reactor is cooled to ambient temperature, the pressure released and a sample is taken for HPLC analysis. A typical oxygen absorption curve and an HPLC trace of the crude MGA is shown in FIG. 10. The A plot shows the reaction, promoted by NaNO₃ while the plot B is recorded in presence of a HNO₃ co-catalyst.

The crude solution of MGA is transferred into a rot evaporator and the solvent acetic acid is removed at 50-55° C. and 20 mm Hg vacuum attained by a water pump. Next, 200 ml of water is added and the rot evaporation continued until the MGA is concentrated to thick viscous syrup. A second analysis is performed to determine the concentration of the MGA and the efficient removal of the acetic acid (CH3COOH). Finally, the crude is diluted to the required concentration with addition of water and stirring in at 40-45° C.

In a typical MGP oxidation reaction using nitric acid (HNO3), instead of NaNO3, the reactor is charged with HNO3 or the total charge is as follows: MGP (43.2 g, 222.5 mmol), AA-TEMPO (0.427 g, 2.0 mmol), acetone (CH3COOH; 109 mL, 114.45 g, 1.90 mol), H2O (1 g, 55.5 mmol) and 5 mL 1M solution of HNO3/CH3COOH (the nitric acid solution is made by diluting 3.144 mL of conc. HNO3 with acetic acid to total volume of 50 mL).

FIG. 11 is a calibration curve for the MGP oxidation in acetic acid. Both an IR and UV detectors were used simultaneously and the two responses were plotted on the same graph. Both area reports were used to calculate the ratio of the RI to UV responses for each peek of interest, eliminating any interference from other by-products. The HPLC analytical conditions were:

Mobile phase—0.005NH₂SO₄

Column—REZEX ROA—Organic Acid (Phenomenex), kept at 40° C.

UV detector—210 nm

RI detector

Total flow rate—0.35 cc/min.

Gradient: None

The sample for the analysis was prepared by taking 0.2 ml aliquots from the reaction solution and dissolving it into 10 mL of the internal standard solution (acetic acid in 0.005NH₂SO₄, 48 mg/mL). Multi level calibration was used to calculate the response factors for MGP, MGA, Oxalic Acid (OA), Tartaric Acid (TA) and Sodium Chloride.

Each MGP oxidation reaction was carried out as follows, unless otherwise stated:

1. Preparing a suspension of Methyl-α-D-Glucopyranoside (MGP) substrate, TEMPO or TEMPO based catalyst, a nitrogen-containing co-catalyst in acetic acid (optionally including water);

2. Heating the stirred reaction solution to the desired temperature under low pressure of oxygen;

3. Pressurizing the system with pure oxygen or air to the targeted process pressure;

4. Monitoring the oxygen uptake and cooling the reaction after the oxygen uptake is completed; and

5. Removing the acetic acid solvent under vacuum by rotary-evaporation. According to one of the purification procedures, the crude solution of the oxidized product (such as Methyl-α-D-Glucuronic acid, or “MGA”) may be transferred into a rotary evaporator and the solvent acetic acid is removed at 50-55° C. and 20 mm Hg vacuum attained by a water pump. Next, 200 ml of water may be added and the rot evaporation continued until the MGA is concentrated to thick viscous syrup. A second HPLC analysis is performed to determine the concentration of the MGA and the efficient removal of the acetic acid. Finally, the crude is diluted to the required concentration with addition of water and stirring in at 40-45° C.

### Example 1

Example 1 shows the activity of an AA-TEMPO/HNO₃ catalyst system at methyl-α-D-glucopyranoside (MGP) to AA-TEMPO ratio of about 150.

The oxidation reactions are carried out in a constant volume, constant pressure volumetric system. The glass autoclave used for these experiments was a 500 mL jacketed reaction flask equipped with a thermocouple, septa fitted addition port and a Teflon coated magnetic stir bar. The reaction flask was connected to an oxygen delivery unit in which gas uptake can be automatically measured and recorded with the progress of the reaction. The reactor is alternately evacuated and purged with oxygen at least five times and the temperature of the catalyst solution was raised to the target temperature at controlled and constant stirring rate.

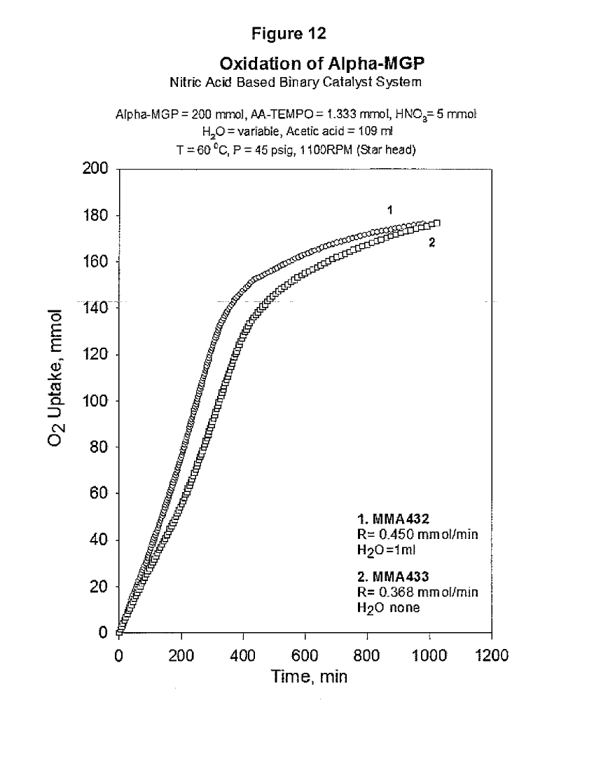

The reaction flask is charged with 38.7 g MGP (199.3 mmol), 284 mg of AA-TEMPO (1.33 mmol), 5 mL of 1M solution of HNO3 in acetic acid, 109 mL acetic acid (1.9 mol) and 1 mL H₂O (55.5 mmol). The stirring is initiated and the thermostating liquid is run into the reactor jacket to bring the catalyst solution temperature to 60° C. The stirring rate in this example was set to 1100 RPM using relatively low efficient stir bar (Teflon coated Star head stir bar). When the temperature reached the target value, the pressure is adjusted to 45 psi and the computer monitoring of the oxygen uptake started. After the consumption of 180 mmol oxygen, which at this reaction conditions is completed in 1000 min, the reactor is cooled to ambient temperature, the pressure released and a sample is taken for HPLC analysis. The graphical presentation of oxygen absorption curve for this reaction is shown in FIG. 12, Curve MMA432. The HPLC analysis of the crude oxidation solution showed 98% conversion of the MGP substrate to MGA at 94% selectivity.

### Example 2

Example 2 is an oxidation reaction of methyl-α-D-glucopyranoside (MGP) similar to the one described in Example 1, but the acetic acid solvent does not contain water as an additive (compare the results with those from Example 1). The graphical presentation of this reaction is shown in FIG. 12, Curve MMA433. The HPLC analysis of the crude oxidation solution showed 98% conversion of the MGP substrate to MGA at 90% selectivity.

### Example 3

Example 3 shows the activity of the binary AA-TEMPO/NaNO₃ based catalyst system at Substrate (methyl-α-D-glucopyranoside) to AA-TEMPO ratio of about 150.

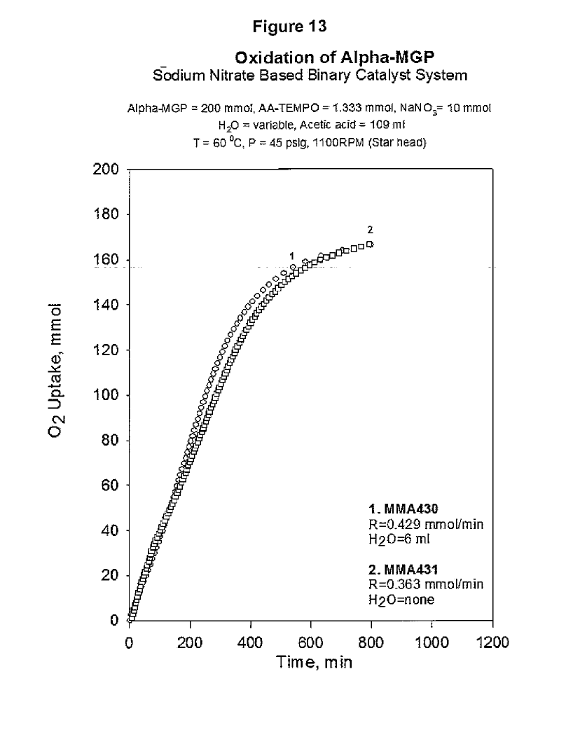

The reaction flask is charged with 38.7 g MGP (199.3 mmol), 284 mg of AA-TEMPO (1.33 mmol), 850 mg NaNO3 (10 mmol), 109 mL acetic acid (1.9 mol) and 6 mL H₂O (333 mmol). The stirring is initiated and the thermostating liquid is run into the reactor jacket to bring the catalyst solution temperature to 60° C. The stirring rate in this example was set to 1100 RPM using Star head stir bar. When the temperature reached the target value, the pressure is adjusted to 45 psi and the computer monitoring of the oxygen uptake started. After the consumption of 180 mmol oxygen, which at this reaction conditions is completed in 800 min, the reactor is cooled to ambient temperature, the pressure released and a sample is taken for HPLC analysis. The graphical presentation of oxygen absorption curve for this reaction is shown in FIG. 13, Curve MMA430. The HPLC analysis of the crude oxidation solution showed 95% conversion of the MGP substrate to MGA at 88% selectivity.

### Example 4

### Example 4 measured an oxidation reaction of methyl-α-D-glucopyranoside (MGP), similar to the one described in Example 3, but the acetic acid solvent does not contain water as an additive (compare the results with those from Example 3). The graphical presentation of this reaction is shown in FIG. 13, Curve MMA431. The HPLC analysis of the crude oxidation solution showed 93% conversion of the MGP substrate to MGA at 80% selectivity.

### Example 5

Example 5 measured the activity of the binary AA-TEMPO/HNO3 based catalyst system at Substrate (methyl-α-D-glucopyranoside) to AA-TEMPO ratio of about 44.

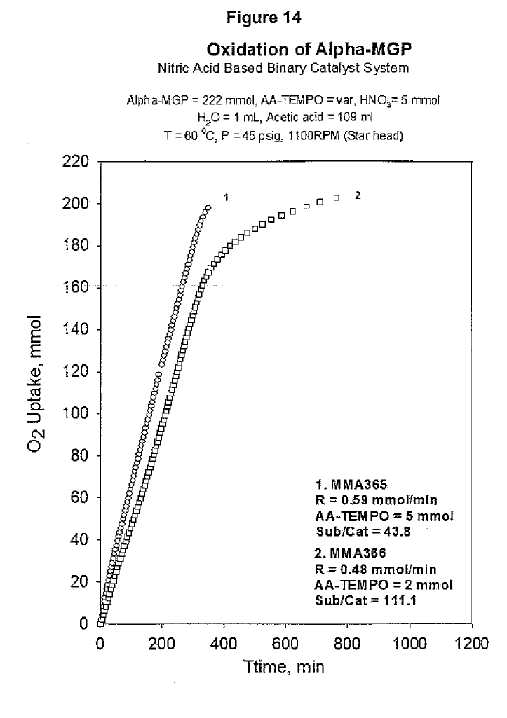

The reaction flask is charged with 43.2 g MGP (222.5 mmol), 1084 mg of AA-TEMPO (5.08 mmol), 5 mL of 1M solution of HNO₃ in acetic acid, 109 mL acetic acid (1.9 mol) and 1 mL H2O (55.5 mmol). The stirring is initiated and the thermostating liquid is run into the reactor jacket to bring the catalyst solution temperature to 60° C. The stirring rate in this example was set to 1100 RPM using Star head stir bar. When the temperature reached the target value, the pressure is adjusted to 45 psi and the computer monitoring of the oxygen uptake started. After the consumption of 200 mmol oxygen, which at this reaction conditions is completed in 400 min, the reactor is cooled to ambient temperature, the pressure released and a sample is taken for HPLC analysis. The graphical presentation of oxygen absorption curve for this reaction is shown in FIG. 14, Curve MMA365. The HPLC analysis of the crude oxidation solution showed 100% conversion of the MGP substrate to MGA at 100% selectivity.

### Example 6

Example 6 represents an oxidation reaction, similar to the one described in Example 5, but the Substrate (methyl-α-D-glucopyranoside) to AA-TEMPO ratio is increased to about 111.

The reaction flask is charged with 43.2 g MGP (222.5 mmol), 427.3 mg of AA-TEMPO (2.0 mmol), 5 mL of 1M solution of HNO3 in acetic acid, 109 mL acetic acid (1.9 mol) and 1 mL H₂O (55.5 mmol). The stirring rate, the reaction temperature and the oxygen pressure are the same as in Example V. The graphical presentation of oxygen absorption curve for this reaction is shown in FIG. 14, Curve MMA366. The HPLC analysis of the crude oxidation solution showed 100% conversion of the MGP substrate to MGA at 96% selectivity.

### Example 7

Example 7 measures the activity of the binary AA-TEMPO/NaNO3 based catalyst system at Substrate (methyl-α-D-glucopyranoside) to AA-TEMPO ratio of about 79.7 and 5 mol % NaNO₃.

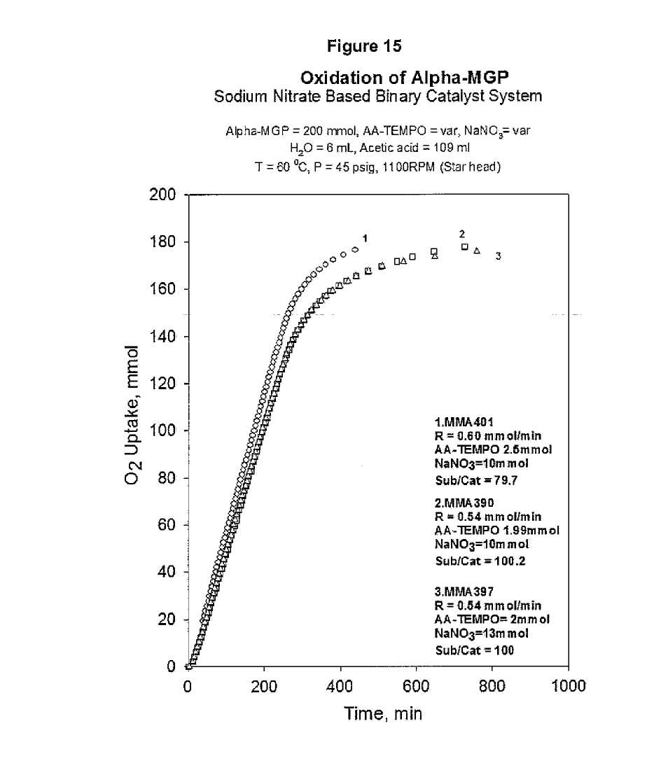

The reaction flask is charged with 38.7 g MGP (199.3 mmol), 533.2 mg of AA-TEMPO (2.5 mmol), 850 mg NaNO₃ (10 mmol), 109 mL acetic acid (1.9 mol) and 6 mL H₂O (333 mmol). The stirring is initiated and the thermostating liquid is run into the reactor jacket to bring the catalyst solution temperature to 60° C. The stirring rate in this example was set to 1100 RPM using Star head stir bar. When the temperature reached the target value, the pressure is adjusted to 45 psi and the computer monitoring of the oxygen uptake started. After the consumption of 180 mmol oxygen, which at this reaction conditions is completed in 450 min, the reactor is cooled to ambient temperature, the pressure released and a sample is taken for HPLC analysis. The graphical presentation of oxygen absorption curve for this reaction is shown in FIG. 15, Curve MMA401. The HPLC analysis of the crude oxidation solution showed 100% conversion of the MGP substrate to MGA at 97% selectivity.

### Example 8

Example 8 measured the activity of the binary AA-TEMPO/NaNO3 based catalyst system at Substrate (methyl-α-D-glucopyranoside) to AA-TEMPO ratio of about 100 and 5 mol % NaNO3.

The reaction flask is charged with 38.7 g MGP (199.3 mmol), 424.3 mg of AA-TEMPO (1.99 mmol), 850 mg NaNO3 (10 mmol), 109 mL acetic acid (1.9 mol) and 6 mL H2O (333 mmol). The reaction conditions are the same as in Example 7. After the consumption of 180 mmol oxygen, which at this reaction conditions is completed in 650 min, the reactor is cooled to ambient temperature. The graphical presentation of oxygen absorption curve for this reaction is shown in FIG. 15, Curve MMA390. The HPLC analysis of the crude oxidation solution showed 100% conversion of the MGP substrate to MGA at 96% selectivity.

### Example 9

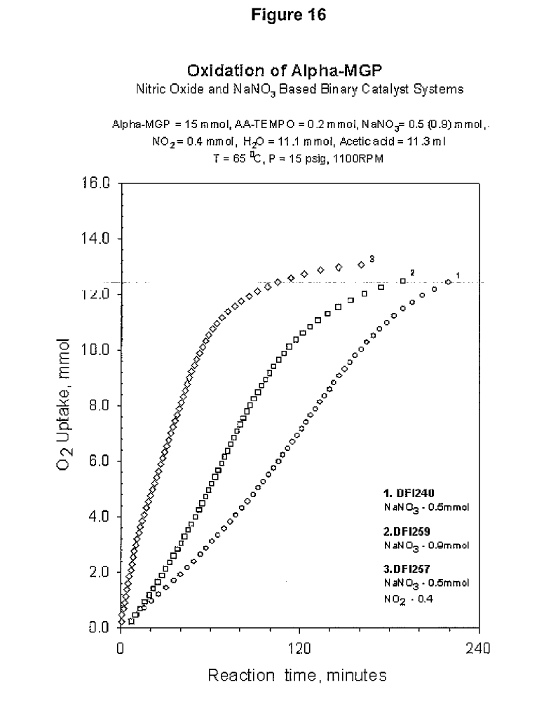

Example 9 is a graph showing the activity of the AA-TEMPO in combination with gaseous NO₂ used as the nitrate source (FIG. 16, curve DFI 257). For comparison, curves DFI240 and DFI 259 are also given to show performance of the AA-TEMPO/NaNO₃ based systems at the same level of nitrate co-catalyst used.

The reaction flask is charged with 2.94 g MGP (15 mmol), 43.5 mg of AA-TEMPO (0.2 mmol), 0.4 mL of 1M solution of NO2 in CH3COOH (0.4 mmol), 0.2 mL of 2.5M solution of NaNO₃ in CH3COOH (0.5 mmol) and 10.9 mL CH3COOH. The stirring rate in this example was set to 1200 RPM using octagon shaped Spin stir bar. When the temperature reached 60 C, the pressure is adjusted to 15 psi and the computer monitoring of the oxygen uptake started. The HPLC analysis of the crude oxidation solution showed 99% conversion of the MGP substrate to MGA at 90% selectivity.

### Example 11

Example 11 measures the activity of the binary AA-TEMPO/HNO₃ based catalyst system at Substrate (methyl-α-D-glucopyranoside) to AA-TEMPO ratio of about 127, at high stirring efficiency and trace amounts of N-Bromosuccinimide.

The reaction flask is charged with 59.0 g MGP (303.8 mmol), 511.9 mg of AA-TEMPO (2.4 mmol), 6 mL of 1M solution of HNO₃ in CH₃COOH, 218 mL CH₃COOH (3.8 mol), 4 mL H2O (222 mmol) and 71.2 mg N-Bromosuccinimide (0.4 mmol). The stirring rate in this example was set to 1200 RPM using octagon shaped Spin stir bar. When the temperature reached 65 C, the pressure is adjusted to 15 psi and the computer monitoring of the oxygen uptake started. The graphical presentation of oxygen absorption curve for this reaction is shown in FIG. 16, Curve MMA439. The HPLC analysis of the crude oxidation solution showed 100% conversion of the MGP substrate to MGA at 95% selectivity.

### Example 12

Example 12 measures the activity of the binary AA-TEMPO/HNO₃ based catalyst system at Substrate (methyl-α-D-glucopyranoside) to AA-TEMPO ratio of about 190, at high stirring efficiency and trace amounts of N-Bromosuccinimide.

The reaction flask is charged with 59.0 g MGP (303.8 mmol), 341.3 mg of AA-TEMPO (1.6 mmol), 4 mL of 1M solution of HNO₃ in CH₃COOH, 218 mL CH3COOH (3.8 mol), 4 mL H2O (222 mmol) and 71.2 mg N-Bromosuccinimide (0.4 mmol). The stirring rate in this example was set to 1200 RPM using octagon shaped Spin stir bar. When the temperature reached 65 C, the pressure is adjusted to 15 psi and the computer monitoring of the oxygen uptake started. The graphical presentation of oxygen absorption curve for this reaction is shown in FIG. 16, Curve MMA440. The HPLC analysis of the crude oxidation solution showed 100% conversion of the MGP substrate to MGA at 96% selectivity.

### Examples 13-17

Examples 13-17 were obtained from a series of liquid phase aerobic oxidations of MGP in a reaction medium containing acetic acid and were carried out in the presence of a catalyst composition comprising AA-TEMPO and a nitrogen-containing co-catalyst (e.g., nitric acid or nitrogen dioxide). Where indicated, the reactions were performed in the presence of a bromide source (e.g., NBS or HBr).

A catalyst composition includes Acetamido TEMPO composition, an N-Bromosuccinimide (NBS) bromide source, and the Methyl-α-D-glucopyranoside (MGP) reactant substrate purchased from Aldrich.

The oxidation reactions were performed using an in-house made Multi Autoclave Glass Volumetric system. All reactions were carried out in Ace Glass reaction flasks with Teflon heads, equipped with Swagelock based injection and thermocouple ports. Digital stirrers and Fisher cross stir bars were used for providing an efficient stirring. The Multi Autoclave reactor system allows conducting five simultaneous oxidations with five independent variables. The reactor system permits the recording of the oxygen uptake with the time, thus allowing precise monitoring the progress of the oxidation.

The reactor medium comprising MGP, Mg(NO₃)₂, AA-TEMPO, acetic acid and/or water was maintained with a bromide source throughout the reaction. The reactor medium was loaded in the reactor and the flask connected to a volumetric manifold. The flask was flushed three times with oxygen and immersed in the thermostated water bath held at 60° C. The oxygen pressure was brought to the required level using the by-pass line and the oxygen admitted at the total process pressure. The continuous monitoring of the oxygen uptake was initiated and recorded against the time. After the reaction was completed, the product composition was analyzed by HPLC using the reaction solvent (acetic acid, CH₃COOH) as an internal standard. Unless otherwise stated, all reactions in this section were run at the following standard conditions: 15 mmol scale (2.97 g MGP), T=60° C., P=45 psi, total solvent volume 11.5 mL at the ratio CH₃COOH (acetic acid):H₂O=10:1.5.

In a typical MGP oxidation reaction using sodium nitrate (NaNO₃), the flask is charged with MGP (38.7 g, 199.3 mmol), AA-TEMPO (0.427 g, 2.0 mmol), NaNO₃ (0.84 g, 10 mmol), acetic acid (CH₃COOH; 109 mL, 114.45 g, 1.90 mol) and H₂O (6 g, 333 mmol). The flask was purged three times with oxygen, pressurized initially to 45 psi and the circulation of the heating liquid was initiated. When the temperature of the reaction mixture reached 60° C., the pressure was adjusted to 45 psi and the computer monitoring of the oxygen uptake started. After the consumption of oxygen, the reactor is cooled to ambient temperature, the pressure released and a sample is taken for HPLC analysis.

The crude solution of MGA is transferred into a rot evaporator and the solvent acetic acid is removed at 50-55° C. and 20 mm Hg vacuum attained by a water pump. Next, 200 ml of water is added and the rot evaporation continued until the MGA is concentrated to thick viscous syrup. A second analysis is performed to determine the concentration of the MGA and the efficient removal of the acetic acid (CH₃COOH). Finally, the crude is diluted to the required concentration with addition of water and stirring in at 40-45° C.

In a typical MGP oxidation reaction using nitric acid (HNO₃), instead of NaNO₃, the reactor is charged with nitric acid and other components of the reaction media as follows: MGP (43.2 g, 222.5 mmol), AA-TEMPO (0.427 g, 2.0 mmol), acetic acid (CH₃COOH; 109 mL, 114.45 g, 1.90 mol), H₂O (1 g, 55.5 mmol) and 5 mL 1M solution of HNO₃/CH₃COOH (the nitric acid solution is made by diluting 3.144 mL of conc. HNO₃ with acetic acid to total volume of 50 mL).

Table 7 discloses certain preferred reaction media and the reaction data for the selective oxidation of MGP to MGA. The table includes the reaction rate (mmol/min), as well as the %-conversion and %-selectivity for the reaction run in each reaction medium (each row corresponds to a single reaction medium and reaction). The reaction media include a MGP reactive substrate and a catalyst composition. The catalyst composition typically includes a nitrogen-containing co-catalyst (NO₂, MgNO₃ or HNO₃), a nitroxyl radical (AA-TEMPO or MeO-TEMPO), acetic acid (“AcOH”) and (optionally) water (“H₂O”). The table shows the millimole (mmol) quantities of the nitrogen-containing co-catalyst and AA-TEMPO nitroxyl radical in the catalyst composition, along with the volume of acetic acid and water in milliliters (mL). Each MGP oxidation reaction in Table 7 was carried out as follows, unless otherwise stated:

1. Preparing a suspension of Methyl-α-D-Glucopyranoside (MGP) substrate, TEMPO or TEMPO based catalyst, a nitrate source co-catalyst in acetic acid (optionally including water);

2. Heating the stirred reaction solution to the desired temperature under low pressure of oxygen;

3. Pressurizing the system with pure oxygen or air to the targeted process pressure;

4. Monitoring the oxygen uptake and cooling the reaction after the oxygen uptake is completed; and

5. Removing the acetic acid solvent under vacuum by rotary-evaporation.

According to one of the purification procedures, the crude solution of the oxidized product (such as Methyl-α-D-Glucuronic acid, or “MGA”) may be transferred into a rotary evaporator and the solvent acetic acid is removed at 50-55° C. and 20 mm Hg vacuum attained by a water pump. Next, 200 ml of water may be added and the rot evaporation continued until the MGA is concentrated to thick viscous syrup. A second HPLC analysis is performed to determine the concentration of the MGA and the efficient removal of the acetic acid. Finally, the crude is diluted to the required concentration with addition of water and stirring in at 40-45° C.

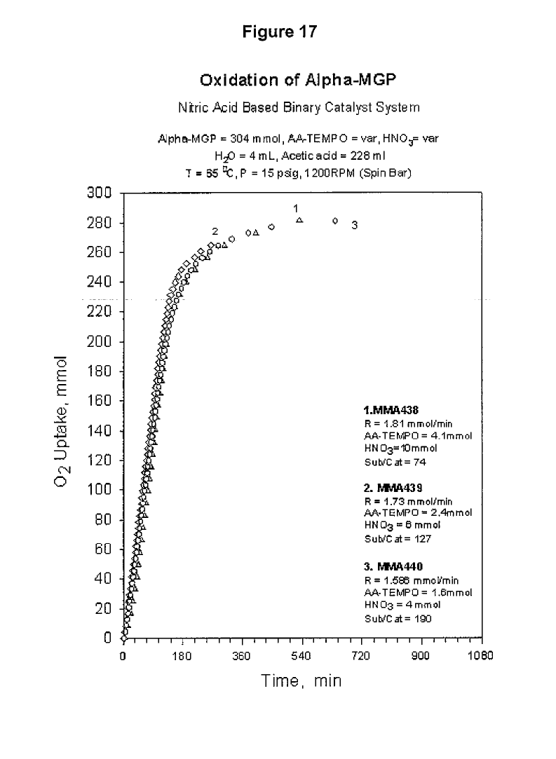

Desirably, the oxidation reactions are performed at conditions permitting a maximum substrate/catalyst ratio while obtaining a desired reaction rate, selectivity and percent conversion. Preferably, substrate/catalyst molar ratios are in excess of 50, 75, 100, 125, 150, 175, 200 or higher. Most preferably, reactant compositions have a substrate/catalyst molar ratio of about 100 or higher, including ratios of about 100-200, 125 and 190. For example, in a first experiment, varying concentrations of nitric acid (HNO₃) were added to a reaction medium consisting essentially of: various amounts of AA-TEMPO nitroxyl radical (4.1 mmol, 2.4 mmol or 1.6 mmol), 304 mmol of the MGP substrate, 228 mL acetic acid and 4 mL water (Examples 13-15 below). The oxidation of MGP was performed at 60 C and 45 psi. oxygen pressure in the reaction vessel. The nitric acid and nitroxyl radical concentrations were varied with and without NBS bromide source. In the presence of 0.4 mmol NBS as a bromide source (curves MMA-439 and MMA-440 in FIG. 17), 100% conversion was achieved at reaction rates of about 1.73 and 1.59 mmol/min were achieved at substrate/catalyst molar ratios of about 127 and 190 and selectivities of about 95% and 96%, respectively. FIG. 17 shows the oxygen uptake curves for the reaction media consisting essentially of the compositions MMA-338, MMA-339 and MMA-440 listed in Table 6 at 65° C. and 15 psig.

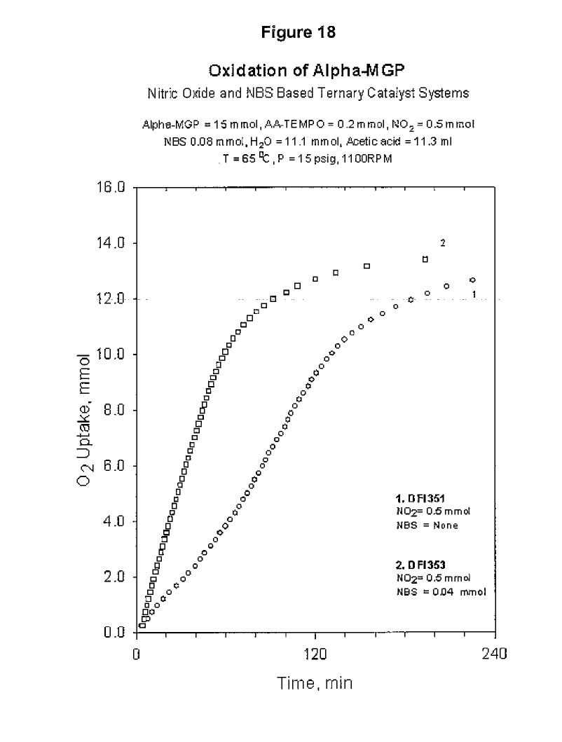

In a second experiment, the nitrate source co-catalyst was nitrogen dioxide (NO₂) in reaction media with and without a NBS bromide source. FIG. 18 shows the oxygen uptake curves for two different reaction media consisting essentially of: 15 mmol α-MOP substrate, 0.5 mmol NO₂ nitrate source, 0.2 mmol AA-TEMPO nitroxyl radical, 11.1 mmol water, 11.3 mL acetic acid and either no NBS or 0.4 mmol NBS bromide source (see Example 16 below). A higher reaction rate was observed when the NBS bromide source was included. Accordingly, a reaction medium comprising nitrogen dioxide preferably includes a bromide source such as NBS. FIG. 18 shows the oxygen uptake curves for the reaction media consisting essentially of the compositions DFI-351 and DFI-353 listed in Table 6 at 65° C. and 15 psig.

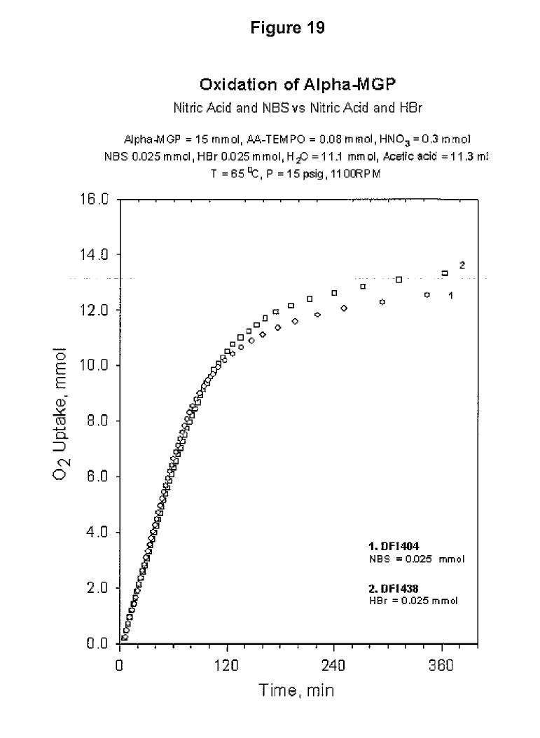

In a third experiment, the nitrate source co-catalyst was nitric acid (HNO₃) in reaction media with one or two different NBS bromide sources: NBS and HBr. FIG. 19 shows the oxygen uptake curves for two different reaction media consisting essentially of: 15 mmol α-MOP substrate, 0.3 mmol HNO₃ nitrate source, 0.08 mmol AA-TEMPO nitroxyl radical, 11.1 mmol water, 11.3 mL acetic acid and either 0.025 mmol NBS or 0.025 mmol HBr as the bromide source (see Example 5 below). A comparable reaction rate was observed when the NBS bromide source was HBr or NBS. FIG. 19 shows the oxygen uptake curves for the reaction media consisting essentially of the compositions DFI-438 and DFI-404 at 65° C. and 15 psig, as listed in Table 6

The oxidation processes can be run as a batch, a semi-batch or as a continuous process, and may be performed in any suitable reactor type or configuration. Thus, stirred tank reactors, tube reactors, reactor cascades, micro reactors or any possible combination of those reactor types might be used.

The oxidation product can be worked up by any known methods such as by phase-splitting, removal of the solvent by distillation, ion exchange or membrane separation techniques. Depending on the requirements for purity, any other chemical methods can also be used. For example, the oxidized product can be subsequently isolated from the reaction media by any suitable method, such as rotary evaporation of the reaction media. For example, the product composition may be analyzed by HPLC using acetic acid (CH₃COOH) as an internal standard.

### Example 13

Example 13 measures the activity of the binary AA-TEMPO/HNO₃ based catalyst system at Substrate to AA-TEMPO ratio of 74, at high stirring efficiency and at lower partial pressure of oxygen.

The reaction flask is charged with 59.0 g MGP (303.8 mmol), 870.0 mg of AA-TEMPO (4.08 mmol), 10 mL of 1M solution of HNO₃ in CH₃COOH, 218 mL CH₃COOH (3.8 mol) and 4 mL H₂O (222 mmol). The stirring is initiated and the thermostating liquid is run into the reactor jacket to bring the catalyst solution temperature to 65° C. The stirring rate in this example was set to 1,200 rpm using octagon shaped Spin stir bar. When the temperature reached the target value, the pressure is adjusted to 15 psi and the computer monitoring of the oxygen uptake started. After the consumption of 280 mmol oxygen, which at this reaction conditions is completed in 500 min (0.56 mmol/min), the reactor is cooled to ambient temperature, the pressure released and a sample is taken for HPLC analysis. The graphical presentation of oxygen absorption curve for this reaction is shown in FIG. 17, Curve MMA438. The HPLC analysis of the crude oxidation solution showed 100% conversion of the MGP substrate to MGA at 97% selectivity.

### Example 14

Example 14 measures the activity of the binary AA-TEMPO/HNO₃ based catalyst system at Substrate to AA-TEMPO ratio of 127, at high stirring efficiency and trace amounts of N-Bromosuccinimide (NBS).

The reaction flask is charged with 59.0 g MGP (303.8 mmol), 511.9 mg of AA-TEMPO (2.4 mmol), 6 mL of 1M solution of HNO₃ in CH₃COOH, 218 mL CH₃COOH (3.8 mol), 4 mL H₂O (222 mmol) and 71.2 mg N-Bromosuccinimide (0.4 mmol). The stirring rate in this example was set to 1200 RPM using octagon shaped Spin stir bar. When the temperature reached 65° C., the pressure is adjusted to 15 psi and the computer monitoring of the oxygen uptake started. The graphical presentation of oxygen absorption curve for this reaction is shown in FIG. 17, Curve MMA439. The HPLC analysis of the crude oxidation solution showed 100% conversion of the MGP substrate to MGA at 95% selectivity.

### Example 15

Example 15 measures the activity of the binary AA-TEMPO/HNO₃ based catalyst system at Substrate to AA-TEMPO ratio of 190, at high stirring efficiency and trace amounts of N-Bromosuccinimide.

The reaction flask is charged with 59.0 g MGP (303.8 mmol), 341.3 mg of AA-TEMPO (1.6 mmol), 4 mL of 1M solution of HNO₃ in CH₃COOH, 218 mL CH₃COOH (3.8 mol), 4 mL H₂O (222 mmol) and 71.2 mg N-Bromosuccinimide (0.4 mmol). The stirring rate in this example was set to 1200 RPM using octagon shaped Spin stir bar. When the temperature reached 65° C., the pressure is adjusted to 15 psi and the computer monitoring of the oxygen uptake started. The graphical presentation of oxygen absorption curve for this reaction is shown in FIG. 17, Curve MMA440. The HPLC analysis of the crude oxidation solution showed 100% conversion of the MGP substrate to MGA at 96% selectivity.

### Example 16

### Example 16 measures the activity of the ternary catalyst composition of AA-TEMPO, NO₂ co-catalyst and NBS (FIG. 18, curve DFI 353). For comparison, the performance of the binary system of AA-TEMPO and NO₂ without NBS is also shown as curve DFI 351 in FIG. 18.

The reaction flask is charged with 2.94 MGP (15 mmol), 43.5 mg of AA-TEMPO (0.2 mmol), 0.5 mL of 1M solution of NO₂ in CH₃COOH (0.5 mmol), 0.2 mL H₂O (11.1 mmol) and 10.8 mL CH₃COOH. For DFI353, the reaction flask also included 0.04 mL of 1M solution of N-Bromosuccinimide in acetic acid (0.04 mmol NBS). The stirring rate in this example was 1,100 rpm. When the temperature reached 65° C., the pressure is adjusted to 15 psi and the computer monitoring of the oxygen uptake started. The HPLC analysis of the crude oxidation solution showed 100% conversion of the MGP substrate to MGA at 93% selectivity.

### Example 17

### Example 17 measures the activity of the ternary catalyst composition of AA-TEMPO, HNO₃ co-catalyst and one of two different bromide sources: a gaseous HBr (FIG. 19, curve DFI 438) or NBS (curve DFI 404 in FIG. 19).

The reaction flask is charged with 2.94 MGP (15 mmol), 0.8 mL of 0.1M solution of AA-TEMPO in CH₃COOH (0.08 mmol), 0.3 mL of 1M solution of HNO₃ in CH₃COOH (0.3 mmol), 1.25 mL of 0.02M solution of HBr in acetic acid (0.025 mmol), 0.2 mL H₂O (11.1 mmol) and 8.95 mL CH₃COOH. The stirring rate in this example was 1,100 rpm. When the temperature reached 65° C., the pressure is adjusted to 15 psi and the computer monitoring of the oxygen uptake started. The HPLC analysis of the crude oxidation solution showed 100% conversion of the MGP substrate to MGA at 91% selectivity.

TABLE 3
Preferred Nitrate Source Catalyst Compositions and Reaction Media Compositions

| | | CATALYST COMPOSITION | | | | | | | |
|---|---|---|---|---|---|---|---|---|---|
| | Substrate | AA | | | | | | | |
| | MGP | TEMPO | Nitrogen-Containing Co-Catalyst (mmol) | | | | | | AcOH |
| FIG. | (mmol) | (mmol) | Mg(NO₃)₂ | KNO₃ | NaNO₃ | KNO₂ | HNO₃ | NO | (mL) |
| 4 | 15 | 0.50 | 0.03 | — | — | — | — | — | 10 |
| 4 | 15 | 0.50 | 0.05 | — | — | — | — | — | 10 |
| 4 | 15 | 0.50 | 0.10 | — | — | — | — | — | 10 |
| 4 | 15 | 0.50 | 0.20 | — | — | — | — | — | 10 |
| 4 | 15 | 0.50 | 0.25 | — | — | — | — | — | 10 |
| 4 | 15 | 0.50 | 0.30 | — | — | — | — | — | 10 |
| 4 | 15 | 0.50 | 0.50 | — | — | — | — | — | 10 |
| 5A | 15 | 0.50 | 0.50 | — | — | — | — | — | 10 |
| 4 | 15 | 0.50 | 1.00 | — | — | — | — | — | 10 |
| 5A | 15 | 0.50 | — | 0.50 | — | — | — | — | 10 |
| 5A | 15 | 0.50 | — | — | — | 0.50 | — | — | 10 |
| 5B | 15 | 0.30 | — | — | — | 0.60 | — | — | 11.5 |

| | | | | MGP: | | | |
|---|---|---|---|---|---|---|---|
| | H2O | MGP: | MGP: | (TEMPO + | TEMPO: | H2O % | AcOH % |
| FIG. | (mL) | TEMPO | NCCC | NCCC) | NCCC | (v/v) | (v/v) |
| 4 | 1.5 | 30.00 | 600.00 | 14.63 | 20.00 | 13.04% | 86.96% |
| 4 | 1.5 | 30.00 | 300.00 | 14.29 | 10.00 | 13.04% | 86.96% |
| 4 | 1.5 | 30.00 | 150.00 | 13.64 | 5.00 | 13.04% | 86.96% |
| 4 | 1.5 | 30.00 | 75.00 | 12.50 | 2.50 | 13.04% | 86.96% |
| 4 | 1.5 | 30.00 | 60.00 | 12.00 | 2.00 | 13.04% | 86.96% |
| 4 | 1.5 | 30.00 | 50.00 | 11.54 | 1.67 | 13.04% | 86.96% |
| 4 | 1.5 | 30.00 | 30.00 | 10.00 | 1.00 | 13.04% | 86.96% |
| 5A | 1.5 | 30.00 | 30.00 | 10.00 | 1.00 | 13.04% | 86.96% |
| 4 | 1.5 | 30.00 | 15.00 | 7.50 | 0.50 | 13.04% | 86.96% |
| 5A | 1.5 | 30.00 | 30.00 | 10.00 | 1.00 | 13.04% | 86.96% |
| 5A | 1.5 | 30.00 | 30.00 | 10.00 | 1.00 | 13.04% | 86.96% |
| 5B | 0.6 | 50.00 | 25.00 | 12.50 | 0.50 | 4.96% | 95.04% |

TABLE 4
Preferred Nitrate Catalyst Compositions and Reaction Media Compositions

| | | CATALYST COMPOSITION | | | | | | | |
|---|---|---|---|---|---|---|---|---|---|
| | Substrate | AA | | | | | | | |
| | MGP | TEMPO | Nitrogen-Containing Co-Catalyst (mmol) | | | | | | AcOH |
| FIG. | (mmol) | (mmol) | Mg(NO₃)₂ | KNO₃ | NaNO₃ | KNO₂ | HNO₃ | NO | (mL) |
| 6B | 15 | 0.30 | — | — | 0.20 | — | — | — | 11.25 |
| 6B | 15 | 0.30 | — | — | 0.30 | — | — | — | 11.25 |
| 6B | 15 | 0.30 | — | — | 0.40 | — | — | — | 11.25 |
| 16 | 15 | 0.20 | — | — | 0.50 | — | — | — | 11.3 |
| 5A | 15 | 0.50 | — | — | 0.50 | — | — | — | 10 |
| 5B | 15 | 0.30 | — | — | 0.50 | — | — | — | 11.5 |
| 6B | 15 | 0.30 | — | — | 0.50 | — | — | — | 11.25 |
| 5B | 15 | 0.30 | — | — | 0.60 | — | — | — | 11.5 |
| 6B | 15 | 0.30 | — | — | 0.60 | — | — | — | 11.25 |
| 16 | 15 | 0.20 | — | — | 0.90 | — | — | — | 11.3 |
| 15 | 200 | 2.50 | — | — | 10.00 | — | — | — | 109 |
| 10 | 200 | 2.00 | — | — | 10.00 | — | — | — | 109 |
| 15 | 200 | 1.99 | — | — | 10.00 | — | — | — | 109 |
| 13 | 200 | 1.33 | — | — | 10.00 | — | — | — | 109 |
| 13 | 200 | 1.33 | — | — | 10.00 | — | — | — | 109 |
| 15 | 200 | 2.00 | — | — | 13.00 | — | — | — | 109 |
| 16 | 15 | 0.20 | — | — | 0.50 | — | — | 0.40 | 11.3 |

| | | | | MGP: | | | |
|---|---|---|---|---|---|---|---|
| | H2O | MGP: | MGP: | (TEMPO + | TEMPO: | H2O % | AcOH % |
| FIG. | (mL) | TEMPO | NCCC | NCCC) | NCCC | (v/v) | (v/v) |
| 6B | 0.25 | 50.00 | 75.00 | 18.75 | 1.50 | 2.17% | 97.83% |
| 6B | 0.25 | 50.00 | 50.00 | 16.67 | 1.00 | 2.17% | 97.83% |
| 6B | 0.25 | 50.00 | 37.50 | 15.00 | 0.75 | 2.17% | 97.83% |
| 16 | 11.1 | 75.00 | 30.00 | 16.67 | 0.40 | 49.55% | 50.45% |
| 5A | 1.5 | 30.00 | 30.00 | 10.00 | 1.00 | 13.04% | 86.96% |
| 5B | 0.6 | 50.00 | 30.00 | 13.64 | 0.60 | 4.96% | 95.04% |
| 6B | 0.25 | 50.00 | 30.00 | 13.64 | 0.60 | 2.17% | 97.83% |
| 5B | 0.6 | 50.00 | 25.00 | 12.50 | 0.50 | 4.96% | 95.04% |
| 6B | 0.25 | 50.00 | 25.00 | 12.50 | 0.50 | 2.17% | 97.83% |
| 16 | 11.1 | 75.00 | 16.67 | 11.54 | 0.22 | 49.55% | 50.45% |
| 15 | 6 | 80.00 | 20.00 | 13.33 | 0.25 | 5.22% | 94.78% |
| 10 | 6 | 100.00 | 20.00 | 14.29 | 0.20 | 5.22% | 94.78% |
| 15 | 6 | 100.50 | 20.00 | 14.31 | 0.20 | 5.22% | 94.78% |
| 13 | 6 | 150.04 | 20.00 | 15.79 | 0.13 | 5.22% | 94.78% |
| 13 | 0 | 150.04 | 20.00 | 15.79 | 0.13 | 0.00% | 100.00% |
| 15 | 6 | 100.00 | 15.38 | 11.76 | 0.15 | 5.22% | 94.78% |
| 16 | 11.1 | 75.00 | 16.67 | 11.54 | 0.40 | 49.55% | 50.45% |

TABLE 5
Preferred Catalyst Compositions and Reaction Media Compositions

| | | CATALYST COMPOSITION | | | | | | | |
|---|---|---|---|---|---|---|---|---|---|
| | Substrate | AA | | | | | | | |
| | MGP | TEMPO | Nitrogen-Containing Co-Catalyst (mmol) | | | | | | AcOH |
| FIG. | (mmol) | (mmol) | Mg(NO₃)₂ | KNO₃ | NaNO₃ | KNO₂ | HNO₃ | NO | (mL) |
| 9 | 15 | 0.30 | — | — | — | — | 0.10 | — | 11.25 |
| 9 | 15 | 0.30 | — | — | — | — | 0.20 | — | 11.25 |
| 5A | 15 | 0.50 | — | — | — | — | 0.25 | — | 10 |
| 9 | 15 | 0.30 | — | — | — | — | 0.30 | — | 11.25 |
| 5B | 15 | 0.30 | — | — | — | — | 0.30 | — | 11.5 |
| 7 | 15 | 0.50 | — | — | — | — | 0.40 | — | 10.25 |
| 7 | 15 | 0.40 | — | — | — | — | 0.40 | — | 10.25 |
| 7 | 15 | 0.30 | — | — | — | — | 0.40 | — | 10.25 |
| 7 | 15 | 0.20 | — | — | — | — | 0.40 | — | 10.25 |
| 7 | 15 | 0.10 | — | — | — | — | 0.40 | — | 10.25 |
| 7 | 15 | 0.05 | — | — | — | — | 0.40 | — | 10.25 |
| 9 | 15 | 0.30 | — | — | — | — | 0.40 | — | 11.25 |
| 5B | 15 | 0.30 | — | — | — | — | 0.40 | — | 11.5 |
| 8A | 15 | 0.30 | — | — | — | — | 0.40 | — | 11.3 |
| 5A | 15 | 0.50 | — | — | — | — | 0.50 | — | 10 |
| 9 | 15 | 0.30 | — | — | — | — | 0.50 | — | 11.25 |
| 8B | 15 | 0.30 | — | — | — | — | 0.50 | — | 11.5 |
| 6A | 15 | 0.50 | — | — | — | — | 0.50 | — | 11.5 |
| 6A | 15 | 0.50 | — | — | — | — | 0.60 | — | 11.5 |
| 6A | 15 | 0.50 | — | — | — | — | 0.70 | — | 11.5 |
| 6A | 15 | 0.50 | — | — | — | — | 0.80 | — | 11.5 |
| 6A | 15 | 0.50 | — | — | — | — | 0.90 | — | 11.5 |
| 14 | 222 | 5.00 | — | — | — | — | 5.00 | — | 109 |
| 10 | 222 | 2.00 | — | — | — | — | 5.00 | — | 109 |
| 14 | 222 | 2.00 | — | — | — | — | 5.00 | — | 109 |
| 12 | 200 | 1.33 | — | — | — | — | 5.00 | — | 109 |
| 12 | 200 | 1.33 | — | — | — | — | 5.00 | — | 109 |

| | | | | MGP: | | | |
|---|---|---|---|---|---|---|---|
| | H2O | MGP: | MGP: | (TEMPO + | TEMPO: | H2O % | AcOH % |
| FIG. | (mL) | TEMPO | NCCC | NCCC) | NCCC | (v/v) | (v/v) |
| 9 | 0.25 | 50.00 | 150.00 | 21.43 | 3.00 | 2.17% | 97.83% |
| 9 | 0.25 | 50.00 | 75.00 | 18.75 | 1.50 | 2.17% | 97.83% |
| 5A | 1.5 | 30.00 | 60.00 | 12.00 | 2.00 | 13.04% | 86.96% |
| 9 | 0.25 | 50.00 | 50.00 | 16.67 | 1.00 | 2.17% | 97.83% |
| 5B | 0.25 | 50.00 | 50.00 | 16.67 | 1.00 | 2.13% | 97.87% |
| 7 | 0.25 | 30.00 | 37.50 | 10.71 | 1.25 | 2.38% | 97.62% |
| 7 | 0.25 | 37.50 | 37.50 | 12.50 | 1.00 | 2.38% | 97.62% |
| 7 | 0.25 | 50.00 | 37.50 | 15.00 | 0.75 | 2.38% | 97.62% |
| 7 | 0.25 | 75.00 | 37.50 | 18.75 | 0.50 | 2.38% | 97.62% |
| 7 | 0.25 | 150.00 | 37.50 | 25.00 | 0.25 | 2.38% | 97.62% |
| 7 | 0.25 | 300.00 | 37.50 | 30.00 | 0.13 | 2.38% | 97.62% |
| 9 | 0.25 | 50.00 | 37.50 | 15.00 | 0.75 | 2.17% | 97.83% |
| 5B | 0.25 | 50.00 | 37.50 | 15.00 | 0.75 | 2.13% | 97.87% |
| 8A | 0.2 | 50.00 | 37.50 | 15.00 | 0.75 | 1.74% | 98.26% |
| 5A | 1.5 | 30.00 | 30.00 | 10.00 | 1.00 | 13.04% | 86.96% |
| 9 | 0.25 | 50.00 | 30.00 | 13.64 | 0.60 | 2.17% | 97.83% |
| 8B | 0.25 | 50.00 | 30.00 | 13.64 | 0.60 | 2.13% | 97.87% |
| 6A | 0 | 30.00 | 30.00 | 10.00 | 1.00 | 0.00% | 100.00% |
| 6A | 0 | 30.00 | 25.00 | 9.38 | 0.83 | 0.00% | 100.00% |
| 6A | 0 | 30.00 | 21.43 | 8.82 | 0.71 | 0.00% | 100.00% |
| 6A | 0 | 30.00 | 18.75 | 8.33 | 0.63 | 0.00% | 100.00% |
| 6A | 0 | 30.00 | 16.67 | 7.89 | 0.56 | 0.00% | 100.00% |
| 14 | 1 | 44.40 | 44.40 | 14.80 | 1.00 | 0.91% | 99.09% |
| 10 | 1 | 111.00 | 44.40 | 24.67 | 0.40 | 0.91% | 99.09% |
| 14 | 1 | 111.00 | 44.40 | 24.67 | 0.40 | 0.91% | 99.09% |
| 12 | 1 | 150.04 | 40.00 | 26.09 | 0.27 | 0.91% | 99.09% |
| 12 | 0 | 150.04 | 40.00 | 26.09 | 0.27 | 0.00% | 100.00% |

TABLE 6
Exemplary Reaction Media Composition and Reaction Data (FIGS. 17-19)

| | | | | | | Rate of | | |
|---|---|---|---|---|---|---|---|---|
| | | AA- | Nitrate | Bromide | | oxygen | | |
| | α-MGP | TEMPO | source | source | Water | consumption | Conversion | Selectivity |
| Curve | (mmol) | (mmol) | (mmol) | (mmol) | (mL) | (mmol/min) | (%) | (%) |
| MMA438 | 304 | 4.1 | HNO₃ | N/A | 4 | 1.81 | 100 | 97 |
| | | | (10 mmol) | | | | | |
| MMA-439 | 304 | 2.4 | HNO₃ | NBS | 4 | 1.73 | 100 | 95 |
| | | | (6 mmol) | (0.4) | | | | |
| MMA-440 | 304 | 1.6 | HNO₃ | NBS | 4 | 1.60 | 100 | 96 |
| | | | (4 mmol) | (0.4) | | | | |
| DFI-351 | 15 | 0.2 | NO₂ | N/A | 0.2 | 0.076 | 100 | 92 |
| | | | (10 mmol) | | | | | |
| DFI-353 | 15 | 0.2 | NO₂ | NBS | 0.2 | 0.268 | 100 | 93 |
| | | | (10 mmol) | (0.08) | | | | |
| DFI-404 | 15 | 0.08 | HNO₃ | NBS | 0.2 | 0.117 | 98 | 88 |
| | | | (0.3 mmol) | (0.025) | | | | |
| DFI-438 | 15 | 0.08 | HNO₃ | HBr | 0.2 | 0.111 | 100 | 91 |
| | | | (0.3 mmol) | (0.025) | | | | |

TABLE 7
Exemplary Reaction Media Compositions and Associated Reaction Data

| | | MeO- | AA- | | | | | | | | Rate | | |
|---|---|---|---|---|---|---|---|---|---|---|---|---|---|
| | MGP | TEMPO | TEMPO | NO2 | | HNO3 | NBS | HBr | AcOH | H2O | (mmol/ | Conv. | Selectivity |
| Sample | (mmol) | (mmol) | (mmol) | (mmol) | MgNO3 | (mmol) | (mmol) | (mmol) | (mL) | (mL) | min) | (%) | (%) |
| DFI006 | 15 | 0.752 | — | — | 0.50 | — | 0.50 | — | 11.5 | 1.5 | 0.366 | 100 | 100 |
| DFI011 | 15 | 0.752 | — | — | 0.50 | — | 0.50 | — | 11.5 | 2.5 | 0.220 | 100 | 100 |
| DFI012 | 15 | 0.752 | — | — | 0.50 | — | 0.50 | — | 11.5 | 3.5 | 0.186 | 100 | 100 |
| DFI013 | 15 | 0.752 | — | — | 0.50 | — | 0.50 | — | 11.5 | 4.5 | 0.101 | 100 | 100 |
| DFI014 | 15 | 0.752 | — | — | 0.50 | — | 0.50 | — | 11.5 | 5.5 | 0.063 | 97 | 100 |
| DFI015 | 15 | 0.752 | — | — | 0.50 | — | 0.50 | — | 11.5 | 6.5 | 0.001 | 4 | 100 |
| DFI026 | 15 | 0.50 | — | — | 0.50 | — | 0.009 | — | 10.0 | 1.5 | 0.146 | 96 | 100 |
| DFI027 | 15 | 0.50 | — | — | 0.50 | — | 0.017 | — | 10.0 | 1.5 | 0.148 | 97 | 100 |
| DFI021 | 15 | 0.50 | — | — | 0.50 | — | 0.025 | — | 10.0 | 1.5 | 0.141 | 100 | 100 |
| DFI022 | 15 | 0.50 | — | — | 0.50 | — | 0.050 | — | 10.0 | 1.5 | 0.168 | 100 | 100 |
| DFI023 | 15 | 0.50 | — | — | 0.50 | — | 0.075 | — | 10.0 | 1.5 | 0.157 | 100 | 100 |
| DFI024 | 15 | 0.50 | — | — | 0.50 | — | 0.100 | — | 10.0 | 1.5 | 0.152 | 100 | 100 |
| DFI025 | 15 | 0.50 | — | — | 0.50 | — | 0.125 | — | 10.0 | 1.5 | 0.141 | 100 | 100 |
| DFI028 | 15 | 0.50 | — | — | 0.2 | — | 0.05 | — | 10.0 | 1.5 | 0.148 | 100 | 100 |
| DFI029 | 15 | 0.50 | — | — | 0.3 | — | 0.05 | — | 10.0 | 1.5 | 0.153 | 100 | 100 |
| DFI030 | 15 | 0.50 | — | — | 0.4 | — | 0.05 | — | 10.0 | 1.5 | 0.131 | 97 | 100 |
| DFI036 | 15 | — | — | — | 1.0 | — | 0.05 | — | 10.0 | 1.5 | 0.000 | 0.0 | — |
| DFI037 | 15 | 0.50 | — | — | 1.0 | — | — | — | 10.0 | 1.5 | 0.109 | 99 | 100 |
| DFI038 | 15 | 0.50 | — | — | — | — | 0.05 | — | 10.0 | 1.5 | 0.000 | 1 | — |
| DFI039 | 15 | — | 0.50 | — | 1.0 | — | — | — | 10.0 | 1.5 | 0.225 | 100 | 100 |
| DFI040 | 15 | — | 0.50 | — | — | — | 0.05 | — | 10.0 | 1.5 | 0.000 | 2 | 100 |
| DFI-351 | 15 | — | 0.2 | 0.5 | — | — | — | — | 11.3 | 0.2 | 0.076 | 100 | 92 |
| DFI-353 | 15 | — | 0.2 | 0.5 | — | — | 0.08 | — | 11.3 | 0.2 | 0.268 | 100 | 93 |
| DFI-404 | 15 | — | 0.08 | — | — | 0.3 | 0.025 | — | 11.3 | 0.2 | 0.117 | 98 | 88 |
| DFI-438 | 15 | — | 0.08 | — | — | 0.3 | — | 0.025 | 11.3 | 0.2 | 0.111 | 100 | 91 |
| MMA-438 | 15 | 4.1 | 4.1 | | | 10.0 | — | — | 7.5 | 4.0 | 1.81 | 100 | 97 |
| MMA-439 | 15 | 2.4 | 2.4 | | | 6.0 | 0.4 | — | 7.5 | 4.0 | 1.73 | 100 | 95 |
| MMA-440 | 15 | 1.6 | 1.6 | | | 4.0 | 0.4 | — | 7.5 | 4.0 | 1.60 | 100 | 96 |

1. A method of selectively oxidizing a portion of a reactive alcohol substrate, the method comprising the step of: reacting a reactant substrate with an oxygen-containing gas in a reaction medium in the absence of a bromide source, the reactant substrate comprising (i) at least one of a primary alcohol moiety or an aldehyde moiety and (ii) a secondary alcohol moiety; the reaction medium containing an organic acid and a catalyst composition, the catalyst composition comprising:
a. a nitroxyl radical; and
b. a nitrogen-containing co-catalyst comprising a molecular species selected from the group consisting of: a nitrate source, nitric oxide and nitrogen dioxide;
the reactant substrate being reacted with the oxygen-containing gas in the reaction medium under conditions of temperature and pressure effective to convert the primary alcohol moiety or the aldehyde moiety to a carboxylic acid moiety in a reaction product having the secondary alcohol moiety within the reaction medium.

2. The method of claim 1, wherein the reactant substrate is a carbohydrate.

3. The method of claim 1, wherein the reactant substrate is a glycol, a primary alcohol, a secondary alcohol, or a sugar.

4. The method of claim 1, wherein the reactant substrate is a sugar alcohol.

5. A method of selectively oxidizing a primary alcohol moiety in a carbohydrate, the method comprising the step of: reacting a carbohydrate reactant comprising a primary alcohol and a secondary alcohol with an oxygen-containing gas in a reaction medium containing a carboxylic acid and a catalyst composition comprising:
a. a nitroxyl radical;
b. a nitrogen-containing co-catalyst comprising a molecular species selected from the group consisting of: a nitrate source, nitric oxide and nitrogen dioxide; and
c. a bromide source,
under conditions of temperature and pressure effective to convert the primary alcohol moiety to a carboxylic acid moiety without oxidizing the secondary alcohol.

6. The method of claim 1 or 5, wherein the reactant substrate comprises a glucopyranose ring.

7. The method of claim 1 or 5, wherein the reactant substrate is an alkyl-α-D-glucopyranoside.

8. The method of claim 1 or 5, wherein the nitrate source comprises a compound selected from the group consisting of: HNO₃, Mg(NO₃)₂, Na(NO₃), and KNO₂, and KNO₃.

9. The method of claim 1 or 5, wherein the nitroxyl radical is selected from the group consisting of: 2,2,6,6-tetramethylpiperidine-1-oxyl, 4-hydroxy-2,2,6,6-tetramethylpiperidine-1-oxyl, 4-oxo-2,2,6,6-tetramethyl-piperine-1-oxyl, 4-amino-2,2,6,6-tetramethylpiperine-1-oxyl, 4-acetamino-2,2,6,6-tetramethylpiperine-1-oxyl, 4-alkoxy-2,2,6,6-tetramethylpiperine-1-oxyl, 4-methoxy-2,2,6,6-tetramethylpiperidne-1-oxyl, and 3,6-dihydro-2,2,6,6-tetramethyl-1(2H)-pyridinyloxy.

10. The method of claim 1 or 5, wherein the reaction medium is further characterized in that:
a. the reaction medium is an aqueous solution comprising about 1%-10% v/v water; and
b. the nitroxyl radical comprises a compound selected from the group consisting of: 2,2,6,6-tetramethylpiperidne-1-oxyl, 4-acetamino-2,2,6,6-tetramethylpiperidne-1-oxyl, and 4-methoxy-2,2,6,6-tetramethylpiperidne-1-oxyl.

11. The method of claim 1 or 5, wherein the reaction medium comprises: 4-acetamino-2,2,6,6-tetramethylpiperine-1-oxyl, acetic acid, and water.

12. The method of claim 1 or 5, further comprising the steps of
a. reacting the reactant substrate in the reaction medium at a temperature of between about 60° C. and about 100° C.; and
b. reacting the reactant substrate in the reaction medium at an oxygen pressure of about 10 psi or higher.

13. The method of claim 1 or 5, wherein the reactant is an alkyl-α-D-glucopyranoside, the nitroxyl radical is 4-acetamino-2,2,6,6-tetramethylpiperidne-1-oxyl, the nitrate source is selected from the group consisting of: HNO₃, Mg(NO₃)₂, Na(NO₃), KNO₃ and KNO₂, and the carbohydrate is reacted with oxygen at a pressure of at least about 10 psi at a temperature of about 60° C. or higher to convert the alkyl-α-D-glucopyranoside reactant to an alkyl-α-D-glucuronic acid.

14. The method of claim 13, wherein the alkyl-α-D-glucopyranoside is methyl-α-D-glucopyranoside and the alkyl-α-D-glucuronic acid is methyl-α-D-glucuronic acid.

15. The method of claim 1 or 5, wherein the catalyst composition comprises:
a. a nitrogen-containing co-catalyst comprising a compound selected from the group consisting of: nitric oxide and nitrogen dioxide; and
b. a nitroxyl radical mediator comprising a compound of formula (I) or formula (II): 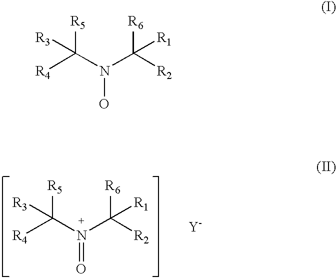
wherein
R¹, R², R³, and R⁴ are independently selected from the group consisting of: a (C₁-C₁₀)-alkyl, a (C₁-C₁₀)-alkenyl, a (C₁-C₁₀)-alkoxy, a (C₆-C₁₈)-aryl, a (C₇-C₁₉)-aralkyl, a (C₆-C₁₈)-aryl-(C₁-C₈)-alkyl and a (C₃-C₁₈)-heteroaryl;
R⁵ and R⁶ are independently selected from the group consisting of: a (C₁-C₁₀)-alkyl, a (C₁-C₁₀)-alkenyl, a (C₁-C₁₀)-alkoxy, a (C₆-C₁₈)-aryl, a (C₇-C₁₉)-aralkyl, a (C₆-C₁₈)-aryl-(C₁-C₈)-alkyl and a (C₃-C₁₈)-heteroaryl; or R⁵ and R⁶ are bonded together via a (C₁-C₄)-alkyl chain, which can be unsaturated or substituted by one or more groups selected from the group consisting of: R¹, C₁-C₈-amido, halogen, oxy, hydroxy, amino, alkylamino, dialkylamino, aryl, diarylamino, alkylcarbonyloxy, arylcarbonyloxy, alkylcarbonylamino and arylcarbonylamino; and
Y is an anion.

16. The method of claim 5, where the carbohydrate reactant is a sugar alcohol comprising a pyranose or furanose ring having a primary alcohol pendant to the ring, and the nitrogen-containing co-catalyst comprises Mg(NO₃)₂, KNO₃, NaNO₃, KNO₂, HNO₃, NO or NO₂ and the carbohydrate reactant is reacted with the oxygen-containing gas at a temperature and pressure effective to convert the primary alcohol moiety pendant to the sugar alcohol to a carboxylic acid moiety without oxidizing the secondary alcohols of the ring.

17. A composition of matter comprising:
a. a sugar or sugar alcohol;
b. a nitroxyl radical;
c. a nitrogen-containing co-catalyst; and
d. an organic acid.

18. The composition of claim 17, wherein the composition is free of a bromide source selected from the group consisting of: NBS and HBr.

19. The composition of claim 17, where the composition further comprises a bromide source.

20. The composition of any one of claims 17-19, wherein
a. the sugar or sugar alcohol comprises a glucopyranose ring.
b. the nitroxyl radical is selected from the group consisting of: 2,2,6,6-tetramethylpiperidne-1-oxyl, 4-hydroxy-2,2,6,6-tetramethylpiperidine-1-oxyl, 4-oxo-2,2,6,6-tetramethyl-piperine-1-oxyl, 4-amino-2,2,6,6-tetramethylpiperine-1-oxyl, 4-acetamino-2,2,6,6-tetramethylpiperine-1-oxyl, 4-alkoxy-2,2,6,6-tetramethylpiperine-1-oxyl, 4-methoxy-2,2,6,6-tetramethylpiperidne-1-oxyl, and 3,6-dihydro-2,2,6,6-tetramethyl-1(2H)-pyridinyloxy;
c. the nitrogen-containing co-catalyst comprises a molecular species selected from the group consisting of: nitric oxide, nitrogen dioxide and a nitrate source comprising a compound selected from the group consisting of: HNO₃, Mg(NO₃)₂, Na(NO₃), and KNO₂;
d. the organic acid comprises a carboxylic acid; and
e. the molar ratio of the sugar alcohol to the nitrogen-containing co-catalyst in the composition is at least about 50.
